(19) United States
(12) Patent Application Publication
(10) Pub. No.: US 2022/0262504 A1
(43) Pub. Date: Aug. 18, 2022

(54) ELECTRONIC ARRANGEMENT FOR THERAPEUTIC INTERVENTIONS UTILIZING VIRTUAL OR AUGMENTED REALITY AND RELATED METHOD

(71) Applicant: Orion Corporation, Espoo (FI)

(72) Inventors: John Raymond Bratty, Nottingham (GB); Christopher Eccleston, Whitley (GB); Sammeli Liikkanen, Espoo (FI); Carina Stenfors, Espoo (FI)

(21) Appl. No.: 17/626,419

(22) PCT Filed: Jul. 10, 2020
(86) PCT No.: PCT/FI2020/050491

§ 371 Date: Jan. 11, 2022

(30) Foreign Application Priority Data
Jul. 12, 2019 (FI) 20195634

(51) Int. Cl. G16H 40/63; G16H 20/30; G16H 20/70

(52) U.S. Cl. CPC G16H 40/63; G16H 20/70; G16H 20/30

(57) ABSTRACT

Electronic arrangement (100) for use in providing therapeutic intervention to a user suffering from a medical condition, optionally to reduce fear of movement and improve function in patients with chronic pain, via virtual reality (VR) or augmented reality (AR), comprising a reproduction equipment (116) comprising a VR and/or AR projection device configured to represent virtual content, comprising an immersive virtual environment or a virtual part of a virtually augmented environment, to the user; user monitoring equipment (114, 114A, 114B) configured to obtain measurement data regarding the user, including motion, location, position, and/or biometric data; and a control system (118, 118A, 118B, 118C), at least functionally connected to the reproduction equipment and the user monitoring equipment, and configured to dynamically determine a personalized therapeutic program including the virtual content for representation via the reproduction equipment, based on the measurement data, wherein the therapeutic program comprises at least two domains of different virtual content, one or more of the domains involving behavior-change con-tent and at least one other domain involving user-activating virtual content indicative of a series of tasks to be conducted by the user having regard to the virtual content through associated therapeutic behavior, such as physical activity or problem solving, in the physical world outside the virtual environment or virtually augmented environment and tracked by the measurement data. A related method is presented.

### FIELD OF THE INVENTION

Generally the present invention pertains to medical technology. In particular, however not exclusively, the invention relates to provision of therapeutic interventions for reduction of pain, such as chronic pain, through utilization of virtual reality (VR) technology. For example, the aim may be in reducing the fear of movement and improving function in patients suffering from chronic pain.

### BACKGROUND

Traditional therapeutic methods, or “interventions”, for at least alleviating symptoms of physical or mental traumas if not actually treating the conditions themselves involve various different challenges. In the case of e.g. physical injury, pain or fear of pain may hinder a subject from conducting day-to-day activities, following a therapeutic rehabilitation program, or simply enjoying life.

Further, with reference to e.g. mental disorders or specifically, anxiety disorders such as generalized anxiety disorder or simple phobias, many of the commonly available pharmacological and non-pharmacological treatment options are not efficacious, or their efficacy is partial, selective or short lived, occasionally reducing the quality of life of a subject to undesired level.

Still, the problems encountered in treating complex medical conditions involving both physiological and psychological aspects, considering e.g. chronic pain, tend to be equally complicated and varied. For example, in a model called the embodied pain framework, chronic disability and distress associated with longstanding pain are considered to be due to a) a privileged access to consciousness of threat-relevant interoception (meaning “bodily sensations are more likely to be attended to, interpreted as threatening, and more likely to be acted upon”), b) avoidance behavior maintained with reinforcement by behavioral consequences of action, and c) subsequent social and cognitive disruption supported by self-defeating behavior and cognition. Treating any of these issues in isolation using traditional methods of therapy has in most cases been found to be sub-optimal.

Yet, in many real-life situations, the provision of traditional types of therapy to address medical conditions such as the ones mentioned or alluded to above, requires interaction between healthcare professional(s) such as therapists, special equipment and a subject simultaneously and contemporaneously in a same space. Fulfillment of these requisites may prove to be difficult if not impossible. Naturally some of these challenges may at least occasionally be overcome by relying upon unsupervised therapy where the subject is expected to take the therapeutic exercises of a therapeutic program prepared for them and handed over to them (e.g. as a paper document) on their own.

However, several issues may emerge also in the context of traditional unsupervised therapy, arising from executing the exercises of a therapeutic program improperly, over exercising or omitting the exercises, for example, which obviously can result in a sub-optimal therapeutic response if not actual additional physiological or mental harm produced to the subject.

### SUMMARY OF THE INVENTION

Thereby, the objective is to alleviate one or more problems described hereinabove not yet fully addressed by the existing arrangements, and to provide a feasible advantageous alternative for providing therapeutic intervention to a user (e.g. patient, subject) suffering from a medical condition.

The objective is achieved by the embodiments of an electronic arrangement and a related method of controlling the arrangement in accordance with the present invention.

In an embodiment, an electronic arrangement, such as one or more at least functionally connected electronic devices, for use in providing therapeutic intervention to a user suffering from a medical condition, optionally to reduce fear of movement and improve function in a user with chronic pain, via virtual reality (VR) or augmented reality (AR), comprises
a reproduction equipment comprising a VR and/or AR projection device, preferably a head-mounted display while also a non-wearable display device could be utilized in some use scenarios, which is configured to represent virtual content, comprising an immersive virtual environment or a virtual part of a virtually augmented environment, to the user;
user monitoring equipment, preferably comprising a number of sensors, configured to obtain measurement data regarding the user, including motion, location, position, and/or biometric data, preferably at least during the consumption of the virtual content by the user, i.e. during a VR/AR session such as a therapeutic session of a therapeutic program; and
a control system, at least functionally connected to the reproduction equipment and the user monitoring equipment, and configured to dynamically determine a personalized therapeutic program including the virtual content for representation via the reproduction equipment, based on the measurement data,
wherein the therapeutic program comprises at least two domains of different virtual content, one or more of the domains involving behavior-change content such as instruction, reflection, relaxation, meditation, behavioral activation, courageous engagement, behavioral reinforcement, fear confrontation, anxiety-causing and/or passive type content, and at least one other domain involving user-activating virtual content, preferably arranged in the form of a rewarded task such as a game incorporating a rewarding and/or scoring aspect (i.e. gamified content) or other stimulating content so as to guide and inspire the user to continue following the therapeutic program, indicative of a series of (mutually identical, similar and/or different) tasks to be conducted by the user having regard to the virtual content through associated therapeutic behavior, including e.g. physical activity and/or problem solving activity, in the physical world (real world) outside the virtual environment or virtually augmented environment and tracked, such as monitored, estimated and/or determined, by the measurement data.

For instance, the arrangement may be configured to obtain an indication of the status, medical condition, and/or selected anthropometric, musculoskeletal or e.g. physiological characteristics of the user, optionally through utilization of the user monitoring equipment and based on measurement data acquired therewith, and to determine the therapeutic program based thereon.

In various embodiments, the user monitoring equipment may comprise e.g. a number of sensors for capturing volitional user input, with reference to optionally user-operable switches, volitional user movement responsive inertial sensors such as accelerometers etc. Yet, the user monitoring equipment may comprise a number of sensors for capturing non-volitional data, optionally comprising various biometric such as vital signs data (e.g. heart rate). Generally, the measurement data may be gathered during the VR/AR experience or specifically the therapeutic program, or outside it. Also subjective (measurement) data such as questionnaire data (answers by the user) may be obtained using e.g. the user monitoring equipment and utilized by and in the context of the present arrangement. Related details are discussed in more detail hereinafter.

In some embodiments, the therapist/healthcare professional may further input user-characterizing (subjective) data such as indication of user status, physical behavior or task performance, detected symptoms such as level of exertion, fear, stress, calmness, or other remarks, e.g. during the VR/AR experience of the user and especially during the execution of the therapeutic program by an input device (e.g. UI device) belonging to the arrangement or being at least functionally connected thereto. These therapist/healthcare professional-provided data and essentially automatically gathered sensor data may be both utilized in the dynamic determination of the therapeutic program. Yet, either of such input data types could be additionally or alternatively used to verify the other, preferably by the arrangement. For instance, human based evaluation data regarding the user could be technically verified, or vice versa based on selected comparison criteria. In case of a sufficient match between the data, e.g. more comprehensive adaptation of the therapeutic program could be triggered in contrast to situations where the two input types of data do not indicate similar user status or performance.

In various preferred embodiments, the personalized therapeutic program provided to the user by the arrangement through a VR/AR experience is dynamically determined such as adapted by the (control system of the) arrangement. Any of the possible elements of the program discussed in more detail hereinelsewhere may thus be dynamically determined.

For example, the user's status, condition, characteristics and/or performance in consuming virtual content, e.g. behavior-change and/or user-activating content, and optionally conducting a related task or series of tasks may be estimated or evaluated e.g. by the measurement data obtained. As the measurement data may indicate e.g. the user's real life (physical world) status, characteristics and/or behavior in a variety of ways, e.g. the measurements-based indication of user behavior may be then evaluated against the therapeutic (user) behavior actually required or desired from the user in the virtual environment or current virtual space thereof, and/or to advance the virtual task(s) provided by the arrangement in said environment or specific space.

In various embodiments, dynamic determination of a therapeutic program may temporally take place already prior to starting the therapeutic program, e.g. during its initial definition or system calibration phase, and/or during the program (during e.g. VR/AR use sessions and/or specifically sessions of the therapeutic program, and/or between them).

For instance, initial or default content of a therapeutic program associated with a user may depend on the medical condition of the user, other status or characteristic information regarding the user and/or initial measurements such as calibration measurements conducted (discussed hereinlater). Yet control information from a healthcare professional may be utilized for program determination (e.g. content selection or other configuration instructions). For a certain medical condition, the arrangement may host and provide a related program including selected type(s) of virtual content in selected proportions and/or order, optionally arranged into one or more therapeutic use sessions, to address the condition, which may refer to alleviating the related symptoms or treating the cause of the condition, for example.

Performance evaluation criteria regarding the therapeutic behavior or its implications in the measurement data may be stored together with the task definition and/or at least linked therewith e.g. in session data or in the therapeutic program. For example, a task or series of tasks may be assigned one or more performance evaluation values such as threshold values as forming at least part of such criteria. Yet, the criteria may include or be at least supplied to general or more specific (e.g. therapeutic program, task, series of tasks and/or session specific) evaluation logic, which may comprise e.g. (value) comparison logic, utilizing the measurement data and evaluation values to determine and output an indication of the performance of the user with a selected resolution. The resolution may be binary (success/failed)) or a finer (e.g. optimal/overdoing/underdoing) one. Based on the result of the performance evaluation, the therapeutic program may be dynamically determined (adapted, for instance).

As indicated above, the therapeutic program comprises user-activating virtual content, advantageously arranged in the form of a rewarded task such as a game, indicative of a series of tasks to be conducted by the user having regard to the virtual content through associated therapeutic behavior, such as movement in the physical world. Yet, there are other type(s) or domain(s) of virtual content as well preferably provided by the arrangement in the therapeutic program, with reference to the afore-mentioned behavior-change content, which may refer to e.g. therapeutic advice or relaxation content.

In various embodiments, e.g. the type, pacing/duration and/or absolute or relative amount of content falling into content domains such as the ones of behavior-change content and user-activating content may be initially determined and/or subsequently adapted by the arrangement e.g. based on the data provided by the user monitoring equipment, such as measurement data acquired by a number of sensors, or data captured otherwise, optionally provided by the user or other person such as a therapist or other healthcare professional.

The arrangement may be configured to alternately or simultaneously provide virtual content from at least these two and/or other possible domains of virtual content of the therapeutic program optionally dynamically and/or as at least part of the dynamic determination of the program. For example, a switchover from certain content to another or if provided simultaneously, their mutual proportions may be dynamically adapted. More detailed examples are provided hereinafter.

In an embodiment of a related method for providing therapeutic intervention to a user suffering from a medical condition by an electronic arrangement through the application of virtual reality (VR) or augmented reality (AR), the method comprises:
providing virtual content comprising immersive virtual environment or a virtual part of a virtually augmented environment to the user via reproduction equipment comprising a VR and/or AR projection device;
obtaining measurement data, via user monitoring equipment, regarding the user, including motion, location, position, and/or biometric data; and
dynamically determining a personalized therapeutic program including the virtual content for representation via the reproduction equipment, based on the measurement data,
wherein the therapeutic program comprises at least two domains of different virtual content, one or more of the domains involving behavior-change content and at least one other domain involving user-activating virtual content indicative of a series of tasks to be conducted by the user having regard to the virtual content through associated therapeutic behavior, such as physical and/or problem solving activity, in the physical world outside the virtual environment or virtually augmented environment and tracked by the measurement data.

Further, it may be provided a computer program product optionally embodied in a preferably non-transitory computer-readable carrier medium such as an optical disc or a memory card comprising instructions, which, when the program is executed by a computer, cause the computer to carry out an embodiment of a method discussed herein. The computer may be included in an embodiment of the arrangement discussed herein. The executing element(s) may include e.g. microprocessor(s) or other controller elements. One or more of the elements may be provided in the control system of the arrangement.

The previously presented considerations concerning the various embodiments of the arrangement may be flexibly applied to the embodiments of the method mutatis mutandis and vice versa, as being appreciated by a skilled person.

The utility of the present invention arises from a plurality of issues still depending on each particular embodiment thereof.

By dynamically optimizing or generally, determining, the therapeutic program as implemented by the embodiments of the arrangement and method of the present invention, including provision of virtual experience including virtual content from preferably multiple content domains responsive to user status or performance as indicated e.g. by user monitoring equipment, various medical conditions may be both efficiently and conveniently addressed remote from therapists or other healthcare professionals, comfortably e.g. at the user's home or other pleasant environment, and related symptoms and/or causes eliminated or at least reduced or managed otherwise in favor of the quality of life of the user.

For example, in terms of pain management, various embodiments of the present invention may provide relief from the pain by the control of executive attention and the rapid shifting to and from pain by the provision of engaging, user-relevant alternative environmental demands. Specifically, but not exclusively, various psychological components (embodiments) of the present solution may be directed at short and long-term treatment of chronic pain and/or its sequellae. In turn, in connection with physiological problems such as physical injuries different embodiments of the present invention may provide a motivating rehabilitation program as well. Yet, the therapeutic programs implemented by embodiments of the present invention may be combinatory in terms of their content and effects, thus addressing both physiological and mental aspects of the user's condition.

Preferred embodiments of the present invention have been also designed safe to use autonomously by the users based on a variety of safety features, i.e. the experience provided to the user has been determined so as to cause no harm or setbacks in the user's condition.

Further, the present invention has been designed to motivate and encourage the users so that they stay engaged with it, which is preferred from the standpoint of implemented therapeutic programs that typically comprise a plurality of use sessions easily spanning an total duration of days, weeks, or months incorporating idle time between the individual sessions, for example. In some embodiments, the overall program may contain a number of modules each associated with certain content and/or tasks (which may optionally have their own/shared objectives in the overall therapeutic program), which may be satisfactorily passed in one or more sessions. In some embodiments, the therapeutic program may be designed without specific overall duration in mind, e.g. for indefinite use.

By various embodiments of the present invention, a realistic VR or AR experience and environment may be provided if desired, closely mapping onto the real world (non-virtual world). Yet, the produced environment and experience may be made immersive. In other embodiments, more artificial look and feel may be preferred from the VR/AR experience, which may still be very immersive. Also mixed solutions are possible (including e.g. more and less realistic content or specifically, sessions or modules of the therapeutic program). By the embodiments, a novel advantageous form of CBT (cognitive behavioral therapy) may be provided to the users to treat e.g. anxiety disorders such as various phobias, through the utilization of e.g. selected behavior-change content. Due to e.g. the level of immersion and potentially realism, the present solution can offer over traditional “office” therapy, a more lasting effect may be attained among the users.

The therapeutic program can also made personal while still working with minimal reflection on how the user should be, feel, or act, i.e. making substantial presumptions on these factors is unnecessary. In various embodiments, selected aspects of gamification and incentivization may be adopted e.g. in the content such as task design and related performance evaluation (evaluation of therapeutic behavior such as physical and/or mental activity required to execute and linked with the task e.g. in task, session or therapeutic program definition). All these features may contribute to the enhanced adoption and engagement of the solution of the present invention by users so that the underlying medical objectives in the light of the conditions the users are suffering from can be addressed to a desired degree.

Through carefully selecting, representing and adapting the virtual content, for example, the above-discussed and other benefits may be achieved. The virtual content may be defined and selected for inclusion in a therapeutic program, or later adapted, in view of e.g. positive reinforcement for goal (objective) and value determined behavior change, guidance on setback planning (overdoing, underdoing, loss of motivation), education (pain without injury, behavior and its consequences), ownership and extension of peripersonal space of the user, and/or motion-related aspects such as extension of e.g. current range of motion among other possibilities.

Items a-c of embodied pain framework were briefly discussed hereinbefore. Accordingly, to cope with such items various embodiments of the present invention may be configured to provide e.g. the following components with content designed to achieve a lasting behavioral change: a) relationship maintenance, b) embodied reactivity, c) courageous engagement, and d) mastery. The content is made achievable preferably within a VR/AR environment creating also a therapeutically designed immersive context: behavioral and computing technology are intelligently blended to achieve a high-impact novel intervention. In particular, an emotionally valent immersive environment can be created by the suggested arrangement, in which the activation of behavioral goals can be safely explored, feared movement can be practiced, peripersonal space can be explored, and self-affirming cognition can become accessible and frequent.

In various preferred embodiments, virtual content or virtual content domains of the therapeutic program may be provided to the user utilizing a number of modes, zones or states, or virtual spaces/sub-environments, which may be mutually clearly distinguishable by the user so that the user can familiarize themselves better and faster with the present invention in favor of user engagement and facilitated use experience, for instance.

A first mode that could be called a ‘personal space mode’, ‘home space’, or ‘safe mode’, for instance, could be created by the help of suitable virtual content as the safe environment or safe space, or similar space for generally managing the VR/AR treatment, giving virtual therapeutic advice and/or distributing relaxation or other behavior-change content. It could be utilized for seeking engagement, setting objectives, choosing domains, reflection and/or relaxation, for example.

A second mode that could be called e.g. an ‘activity space mode’ could be provided to execute, for instance, the active therapeutic treatment involving the series of tasks to be conducted by the user and preferably relying upon principles of gamification and incentivization as discussed in more detail hereinafter. Through immersion and appropriate content design the user's internal and intuitive decision making and incentivization processes may be targeted and reached in favor of e.g. desired behavioral changes.

Both the behavior-change and user activating content have been found useful in treating different medical conditions. As discussed herein, both content types could be provided via the same virtual space or environment simultaneously, and/or at different times and/or via different virtual spaces or modes. Yet, the interaction between different modes and/or related content types, and related transitions have been found beneficial in achieving the desired therapeutic objectives.

Dynamic determination of a therapeutic program delivered by the embodiments of the present invention may include dynamically determining or specifically adapting practically any element of the program such as the virtual content, data acquisition via the user monitoring system or associated data processing, and/or criteria applied e.g. for task evaluation so that it becomes progressive. In other words, the user's performance in conducting the tasks assigned to them and/or other real-world data obtained during or outside of the VR/AR sessions may be configured to have a selected impact on how e.g. the content defining the therapy and e.g. support in it are introduced to the user over time. Adaptation of virtual content or a therapeutic program in general may optionally involve utilization of one or more selected algorithms such as AI algorithms or specifically, machine learning instead of or in addition to more fixed or linear operation logic.

The suggested solution may further be realized as an electronic arrangement of at least functionally, such as communications-wise, connected group of devices, which may include e.g. VR/AR reproduction gear such as a headset, user monitoring equipment such as specific controller devices, sensor devices, or multipurpose devices such as personal communication devices (e.g. smartphones) that are harnessed into providing data about the user, and a control system such as one or more computer devices.

As being easily comprehended by a person skilled in the art, these components may be, depending on the embodiment, physically integrated rather flexibly and selectively as well. For example, various elements of the arrangement may be integrated concerning e.g. a headset, which could contain at least part of the control system (e.g. processing unit(s), memory and/or communication interface towards remote parts of the arrangement or external systems/devices) and/or user monitoring equipment (e.g. sensors) as well. In some embodiments, the suggested solution includes local elements or local sub-system substantially at the location of the user and at least one remote sub-system functionally connected to the local sub-system. Use of both local (e.g. more portable, affordable, personal, simpler, compact, less power consuming, easier to attain, etc.) and remote entities may be capitalized so as to make the VR/AR experience and related therapeutic treatment more robust, dynamic, adjustable, personalized and holistic, for instance, instead of e.g. simpler purely local standalone solution. More complex and extensive and/or less urgent calculations could be performed at a computationally more efficient or flexible (e.g. cloud computing platform based) remote system(s) while the local, perhaps computational resources-wise also lesser, system may be capable of more rapid action or initial action (or e.g. personalize more generic data obtained from remote system(s) for the particular local user) while still preferably being also capable of autonomous action and advantageously at least limited independent adaptation to prevailing circumstances as indicated by the measurement data e.g. in situations where communication connections between the systems are limited.

In various preferred embodiments, as the measured data indicative of e.g. the user's performance in conducting the tasks provided to them optionally in a gamified form may be combined with selected non-game/non-VR/AR real world data potentially also obtained outside of therapy or generally outside VR/AR sessions to adapt the virtual content or the therapeutic program in general as provided by the electronic arrangement of the present invention, a remarkably holistic solution may be achieved with increased accuracy as to the evaluation capability of the user's current status, other characteristics and progression in terms of the therapeutic program for duly treating the concerned medical condition. The real-world data may include e.g. sleep or particularly insomnia related data (and indeed, e.g., sleep or sleep related characteristics may be improved by various embodiments of the present invention), overall activity or passivity data, stress, anxiety and/or other measurements either done automatically by a number of sensors or obtained using other data collection methods which can also be both subjective or objective, with reference to e.g. questionnaires answered by the users.

In the context of treating various medical conditions, e.g. chronic or long-lasting pain and/or e.g. motion related disabilities such as a limited range of motion of a limb, virtual content that is easy to understand and adopt by the users while still enabling complex and inspirational or motivational enough applications in the light of tasks to be conducted through their manipulation in accordance with basic principles of the present invention, has been found important. Successful execution and completion of tasks may involve different aspects of problem solving and/or physical activity such as movement. Use of certain geometric shapes such as tetrominoes in the virtual content e.g. as virtual target objects of tasks have been found particularly feasible. Such geometric shapes may be virtually represented to the user in a gamifi(cat)ed fashion so that the user may, by real-life actions such as movement of their hands or limbs in general, conveniently address and manipulate them as desired (translational movement, rotation, etc.) in the light of the task objectives (goals) that may involve e.g. piling the shapes and/or constructing an instructed overall shape therefrom. Thus, the execution of both coarser and finer motoric actions may be required, in addition to e.g. (mental) problem solving, from the user as therapeutic behavior, e.g. in an adaptable ratio, in order to successfully conduct the task. Still, the user conveniently remains well engaged with the stimulating overall task and there is no need, for example, to separate the actions into different exercises or sessions. Several aspects requiring slightly different skills considered beneficial for treating the user's condition may thus be cleverly combined into a single greater task and/or session/module.

Yet, the results achieved by various embodiments of the present invention may be clinically and technically reliably assessed even in real-time fashion and the condition of the user monitored besides prior to or during the actual VR/AR therapy or related therapeutic program, also by subsequent follow-ups, for instance. Various technical equipment such as the monitoring equipment and/or control system of the arrangement of the present invention and/or systems/devices functionally connected therewith may be still applied for the purpose. Remote monitoring of the user is thus feasible as well. Subjective data may be further gathered from the user e.g. via their personal electronic devices such as smartphones or other terminals. The same and/or different measurements than already conducted prior to or during the VR/AR therapeutic program may also be utilized in the evaluation of the outcome of the treatment and for post-treatment monitoring activities. Based on the data provided and optionally further analyzed by the follow-up(s), a number of responsive actions may be triggered. For example, healthcare professionals may be provided with follow-up reports and alarms preferably by the arrangement depending on the user's status, such as location, pain and/or (range of) movement related status, as indicated by the data. The user may be contacted and/or a new therapeutic program assigned to them for execution if a need arises (if the user's condition is getting worse based on the data, for example).

Discussion regarding the utility of the embodiments of the present invention is continued below in the detailed description.

The terms “psychological” and “mental” are utilized interchangeably in this text unless explicitly cited otherwise.

The terms “motion” and “movement” are utilized interchangeably in this text unless explicitly cited otherwise.

The term “healthcare professional” may herein refer, for instance, to a therapist, physician, medical doctor, pharmacist, physiotherapist, nurse, medical assistant or any other person who utilizes an embodiment of the present invention, or a device or system connected thereto as implied in this text, for monitoring, instructing, communicating with or otherwise addressing the user from a therapeutic standpoint notwithstanding the fact whether the person is formally registered or not as a healthcare professional according to the local rules and regulations of a region wherein the present invention is utilized.

The numerals “first” and “second” are utilized herein to distinguish an element from another element, e.g. left hand from right hand, and not to prioritize between the elements or arrange them in any particular order unless otherwise explicitly cited.

The expressions “a number of” and “series” refers herein to any positive integer starting from one (1), e.g. to one, two, three, or more.

The expression “a plurality of” refers herein to any positive integer starting from two (2), e.g. to two, three, four, or more.

### BRIEF DESCRIPTION OF THE RELATED DRAWINGS

Next the invention is described in more detail with reference to the appended drawings in which

FIG. 1 is a block diagram of an embodiment of an electronic arrangement in accordance with the present invention and possible connected systems or devices.

FIG. 2 illustrates one typical use scenario of the arrangement in accordance with an embodiment of the present invention.

FIG. 3A illustrates a view incorporating behavior-change such as relaxation type virtual content in the form of a virtual space and related virtual objects, which could be provided to the user via the reproduction equipment of an embodiment of the arrangement of the present invention. Behavioral therapy is preferably provided in this space or e.g. by display content or audio (e.g. voice) content.

FIG. 3B illustrates a more focused snapshot taken from alternative position in the virtual environment and space depicted in FIG. 3A.

FIG. 4A illustrates a view such as display view of virtual content comprising user-activating content that may be provided to the user via the reproduction equipment.

FIG. 4B illustrates a further view of virtual content such as fear confrontation type behavior-change content.

FIG. 5 illustrates still a further view combining different virtual content.

FIG. 6A illustrates an embodiment of a therapeutic program including virtual content and guidelines for evaluating the performance of a concerned user.

FIG. 6B illustrates an embodiment of dynamically determining (adapting, selecting, defining etc.) the therapeutic program as provided by the electronic arrangement in terms of e.g. pacing/duration thereof in response to e.g. related control input obtained.

FIG. 7 is a flow diagram of an embodiment of a method in accordance with the present invention.

FIG. 8 Sample data from trial described in Example 1. One position axis of the left controller over the time.

FIG. 9 Sample data from trial described in Example 1. Left controller distance (in 3D space) from headset (used to detect single movements) over the time (seconds) along two speed vectors.

FIG. 10 Sample data of from trial described in Example 1. Selected time frame (40 seconds) from left controller distance from headset showing the detected movements (push or draw) during the time frame.

FIG. 11 Sample data of from trial described in Example 1. Single detected movement of a pain subject.

FIG. 12 Sample data of from trial described in Example 1. Single detected movement of a healthy subject which show less variance in speed i.e. the movement is much more harmonious.

### DETAILED DESCRIPTION OF THE EMBODIMENTS

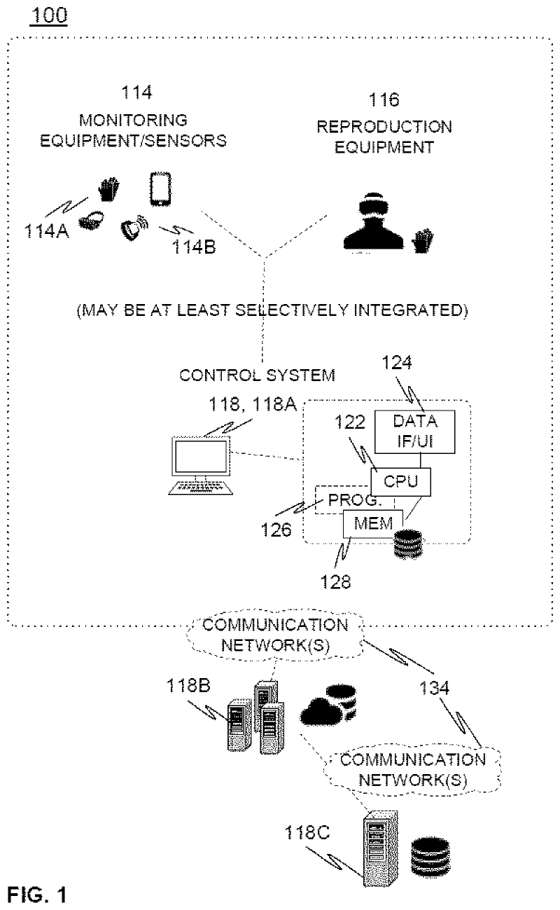

FIG. 1 shows, at 100, a block diagram of an embodiment of an electronic arrangement in accordance with the present invention. Depending on the implementation, the arrangement may be considered or implemented as a device or a system of at least functionally such as communications-wise connected devices.

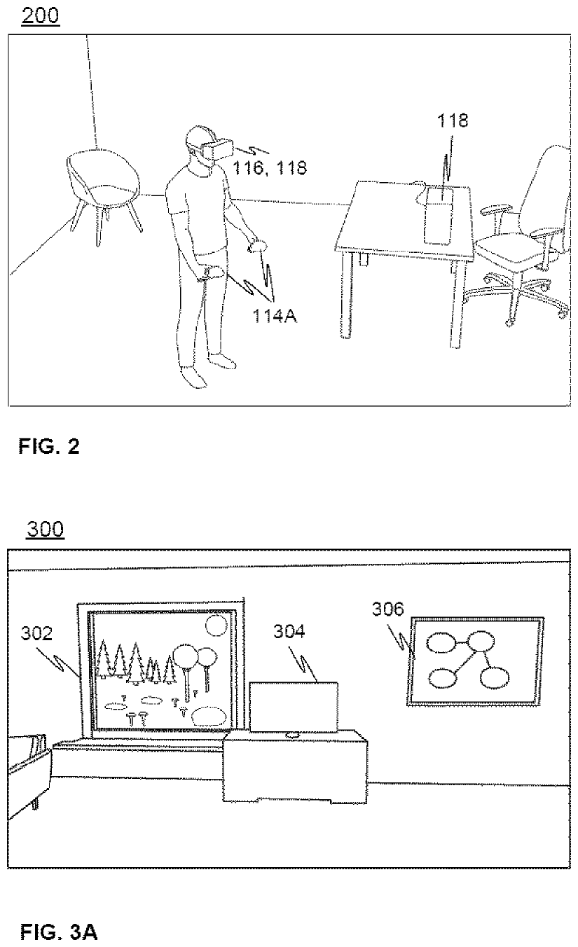

In the use scenario 200 of FIG. 2, a user 201 (patient, subject) is shown wearing a headset that hosts e.g. at least part of reproduction equipment 116, and potentially also of user monitoring equipment 114 and/or control system 118. The user 201 is enjoying the VR/AR experience e.g. at their home or in other comfortable or suitable environment (e.g. familiar, peaceful, and/or safe environment) typically but not necessarily remote from e.g. actual healthcare facility such as therapist's or other healthcare professional's appointment. The concerned location could also refer to e.g. a gym or other venue where the user 201 is rehabilitating and/or exercising with equipment such as an exercise bike locally available thereat in which case the arrangement could be utilized to render the exercises more meaningful or inspiring, efficient and/or less painful, for example.

Depending on the particular use scenario and embodiment of the arrangement, the arrangement or certain elements thereof may serve a single user or multiple users as being discussed in more detail hereinafter.

The arrangement may, as a whole, be physically implemented by at least one electronic device, and more typically, however, by multiple functionally such as communications-wise connected electronic devices as already briefly mentioned above. Nevertheless, when considering the internals of the arrangement at least from a functional standpoint, reproduction equipment 116, user monitoring equipment 114 and a control system 118 (in more limited or comprehensive form, which is discussed later) can be identified and advantageously included therein. The equipment 114, 116 is advantageously configured and controlled so as to implement a target UI (user interface) to the arrangement from the standpoint of the user 201. The resulting UX (user experience) may be dynamically adapted based on e.g. obtained measurement data and/or selected contextual factors such as location of the user, weather, time (of day), etc., which may be technically monitored as well, optionally by one or more elements of the equipment 114 or by external devices still functionally connected to the arrangement, as being easily understood by a person skilled in the art.

The reproduction equipment 116, which may include commercially available equipment/hardware and/or proprietary gear, may be configured to reproduce virtual content of e.g. different content domains to the user as already mentioned hereinbefore. In addition, the arrangement may be configured to store and indicate, preferably via the reproduction equipment 116, the user's performance in meeting an objective such as conclusion of tasks to be performed preferably against the user's previous performance and/or the performance of a number of other users. Execution of the tasks in virtual or virtually augmented space, or generally environment, may require therapeutic such as physical behavior associated therewith to take place in the physical world (i.e. real world, non-virtual world), with reference to e.g. physical activities such as motion. The virtual content may further include content such as behavior-change content of various types. This content may be associated with tasks requiring more psychological type therapeutic behavior such as certain level of calmness, peacefulness, bravery or fearlessness (may be to certain extended technically monitored by sensors) instead of or in addition to physical activity to take place in the real world, regarding e.g. fear confrontation. The tasks provided to the user may generally involve various aspects of problem solving.

The reproduction equipment 116 may include, e.g. in favor of increased immersion, at least one element selected from the group consisting of: VR headset, AR headset, combined VR-AR headset, display, audio speaker, haptic feedback providing device, vibration device, scent-generating device (scent data may be included in the virtual content data), and wearable haptic feedback providing device (e.g. glove(s), ring(s), vest). Naturally e.g. the headsets may comprise e.g. one or more displays for visual content provision and preferably also one or more speakers (at least one for each ear, for example) for audio content output.

A haptic device may be configured to provide a haptic sensation of touch (pressure) to the user preferably at least responsive to contacting a virtual object such as a wall or e.g. some specific target object to be manipulated in the virtual or augmented environment. Yet, the haptic device may comprise a counter force mechanism to simulate e.g. physical workload such as lifting, pulling, pushing or otherwise interacting with (physical) objects.

The user monitoring equipment 114 is typically configured to obtain measurement data concerning the user, which may refer to control input provided by the user as well as a multitude of other information characterizing the user, for instance.

The user monitoring equipment 114 may comprise one or more pieces of equipment 114A that are intended for use, either solely or at least, during the overall VR/AR experience and/or specifically during related VR/AR therapeutic sessions (i.e. during actual therapy). Such equipment 114A may include e.g. dedicated controller(s) such as hand(-held) controller(s), headset-integrated sensor(s) such as inertial sensor(s), position/location sensor(s) and/or other wearable controller(s)/sensor(s) for providing user input during VR/AR session. Indeed, various user input devices such as hand controllers may generally comprise one or more sensors for registering volitional user/control input and optionally providing other measurement data.

As indicated above, at least some of the monitoring equipment 114 may be integrated with reproduction equipment 116 e.g. in a headset type of an apparatus (e.g. inertial sensors, (other) position/location sensors, camera (e.g. image data may be utilized for user distance/location/position estimation, etc.), one or more microphones).

Yet, at least some of the equipment 114 such as (one or more hand) controller(s) may be specifically utilized at least for interacting with such as navigating in the VR/AR realm and related content.

In more detail, the arrangement and specifically e.g. control system 118 therein may be configured to alter the user's, or of a corresponding virtual character's or pointer's, position, location, rotation or translational speed, and/or viewing direction in a virtual environment (or virtually augmented environment when applicable) such as virtual room or other virtual space based on the measurement data such as data indicative of the user's volitional control input captured through one or more sensors of the user monitoring equipment.

Additionally or alternatively, the arrangement or specifically e.g. control system 118 therein may be configured to adapt one or more virtual objects illustrated in a virtual environment or virtual part of the virtually augmented environment, optionally type, size, color, rotation, movement, position, and/or location, based on the measurement data such as the aforementioned volitional control input or other measurement data such as sensor data indicative of e.g. any of vital signs or other status/characteristic information concerning the user. As an example of the former, the user 201 may, for example, grab a virtual object in the virtual space/environment by executing a physical action such as grabbing action in the real world, linked with the grabbing activity in the virtual environment.

The equipment 114A may include e.g. commercially available mobile and/or wearable, optionally implantable, devices comprising different sensors for e.g. motoric and non-motoric data collection during the VR/AR sessions or specifically during therapeutic events or activities such as execution of tasks ordered to the user. The equipment 114, 114A typically communicates with the control system 118 of the arrangement.

Yet, the user monitoring equipment 114 may comprise one or more pieces of equipment 114B that is intended for use, either solely or at least, during periods excluding the VR/AR sessions or at least actual VR/AR therapy. The equipment 114B may comprise e.g. a personal, optionally portable, computer or personal portable communications (terminal) and/or multimedia device such as a smartphone or a wristop device such as a smartwatch among numerous other options such as implantable sensor(s). The equipment 114B may be configured to communicate with the control system 118 of the arrangement and/or e.g. with entity 118C discussed in more detail hereinlater without necessarily having other elements of the arrangement in between on the communication path, still depending on the particular element or device of the equipment 114B in question. The equipment 114B may further include a number of devices such as sensor devices that are not actively personally worn or carried by the user while being potentially fixed at target location(s), with reference to e.g. sensor(s) located at one's home, other location(s), or e.g. vehicle(s), furniture or other physical object(s), considering e.g. a sensor for door (e.g. room/building/cabinet/fridge), room/space, surroundings, etc.).

Indeed, in various embodiments the arrangement may also be configured to obtain real-life measurement data of subjective and/or objective nature regarding the user during periods outside the consumption of the virtual content. The obtained measurement data may comprise at least one data element selected from the group consisting of: user activity information, call data, messaging data, SMS (short message service) or multimedia messaging data, communication data, internet data, used search term, physical activity or passivity data, sleep, insomnia or other sleep disorder related data, social activity data, social media activity data, motion, motoric, location, position, user reported data, pain data, and/or biometric data. This data may be utilized jointly with the data gathered during VR/AR sessions to analyze the user's status and characteristics, for instance. Yet, such measurement data indicative of e.g. status, characteristics, location and/or movements of the user may include or be at least associated with contextual information (toilet visits per time duration such as night hours or use times of a fridge, for example), which may be technically monitored as being appreciated by a person skilled in the art utilizing many different sensors (e.g. microswitch in a selected door, camera/motion sensor in a selected space, etc.) that are preferably at least functionally connected to the arrangement.

As alluded to above, in addition to potentially fully automated acquisition of e.g. sensor-based measurement data the arrangement may be configured to obtain, optionally still via the user monitoring equipment 114 (114A and/or 114B) either during or outside VR/AR experience, or at least VR/AR therapy, typically but not exclusively user-created more subjective (measurement) data such as questionnaire data (answers), validated questionnaire data, non-validated questionnaire data, PROM (patient reported outcome measures) questionnaire data, Tampa Kinesiophobia Scale (TKS) questionnaire data, the Oswestry Scale questionnaire data, PASOL (pain solutions questionnaire), ECID (Experience of Cognitive Intrusion of Pain), EQ-5D™ or other scale of e.g. movement related questionnaire data, insomnia scale questionnaire data, pain questionnaire data, quality of life questionnaire data, digital notes or diary data, therapeutic professional provided data (based on monitoring the user during e.g. the VR/AR session or specifically during the execution of therapeutic program/related task either remotely or on the spot), and/or discussion data (indicative of e.g. messaging or other communication taken place via the arrangement or a connected system, between the user and another entity or specifically a person, such as a therapist/healthcare professional (real person or virtual)), wherein the obtained data preferably characterizes e.g. the status or condition, such as mental or physical condition, of the user 201, or their performance. For example, mood, feelings, pain and/or other symptoms may be indicated in the subjective data either in response to specific queries by the arrangement or autonomously by the user. The data may be utilized by the arrangement in dynamically determining the personalized therapeutic (treatment) program, such as at least a component like a session or module thereof or e.g. task or content item associated with the session/module/program, for example. Utilization of the data may comprise analyzing it via selected processing technique(s) such as mapping or filtering.

In some embodiments, the aforementioned questionnaires may include self-administered questionnaires. These questionnaires could be arranged on paper but are more preferably executed digitally via a user terminal such as a computer device, a smartphone or smartwatch, or e.g. VR/AR headset and/or other device of the arrangement. Such assessments may be made on a regular basis such as weekly or daily, and/or responsive to e.g. an occurrence of a specific triggering event such as user activation of new content via the arrangement or user enrollment/registration.

Yet, in some embodiments, the arrangement is configured to (indirectly) estimate the user's psychological status such as affect, motivation, cognition or behavioral intention. The (measurement) data used for the purpose may include e.g. search terms of information searches such as so-called internet (search engine) searches or social media/messaging service/message entries (used terms/entries may be mapped to an estimate), whereas e.g. the rate of power consumption/battery consumption of a user terminal will reveal general phone time ((social) activity), the positioning data such as GPS data will reveal the amount or type of exercise or generally movement in space and/or e.g. the nature or destination of trips taken (based on comparison with available map/location data regarding e.g. commercial venues) such as shopping trips, and the time of trips will reveal e.g. temporal patterns of planned or unplanned behavior in the context of e.g. pain.

Evaluation criteria for assessing or deriving the meaning of the obtained data such as internet search terms from the standpoint of the present invention may be stored in the arrangement in the form of evaluation logic and/or data mapping structures such as data tables, for instance.

Generally, the user monitoring equipment 114, 114A, 114B may include commercially available and/or proprietary electronic devices such as mobile and/or wearable devices for data acquisition, the devices being potentially equipped with different sensors for e.g. motoric and non-motoric data collection.

In terms of included sensors or sensing functionalities, the user monitoring equipment 114, 114A, 114B may comprise at least one element selected from the group consisting of: control input sensor (e.g. a user-operable switch or other explicit user input providing element), inertial sensor, motion sensor, accelerometer, wearable inertial sensor such as accelerometer, limb-attachable or hand held inertial sensor such as accelerometer, gyroscope, camera, optical sensor, pressure sensor, temperature sensor, moisture sensor, distance sensor, eye sensor, position/location sensor, positioning signal (e.g. satellite such as GPS and/or local) receiving sensor, biometric sensor, microphone, and controller or reproduction equipment included such as headset included sensor. The controller or reproduction equipment included sensors may comprise e.g. accelerometer or other inertial sensor, camera or microphone. In terms of e.g. inertial sensors, one or multi-axis sensors may be utilized. In some embodiments location/position sensing could be based on pressure sensing instead of or in addition to other options such as inertial or positioning signal receiving sensors, with reference to e.g. a carpet or garment.

Potential quantities to measure by various embodiments of the arrangement preferably include e.g. at least one element selected from the group consisting of: motion, location, position (e.g. alignment or heading), acceleration (/deceleration), velocity, speed, motion of a selected target element such as controller, head, neck, limb (arm(s) and/or leg(s)) or trunk, location of a selected target element such as controller, reproduction device, head, neck, limb or trunk, position of a selected target element such as controller, reproduction device, head, neck, limb or trunk, speed, velocity or acceleration (deceleration) of a selected target element such as controller, reproduction device, head, neck, limb or trunk, and biometric data such as vital signs data regarding the user.

For example, location or position data may be indicated using any of the three Cartesian coordinates (x,y,z) relative to a used reference.

Measurements may be executed using regular predefined or adaptive sampling or generally measurement rates (e.g. a higher rate used with tasks requiring more precise evaluation and/or more rapid action, and vice versa), such as rates ranging from about one or few Hz to few kHz, for example, preferably at least during the consumption of the virtual content by the user or more specifically, during an execution of task evaluation of which benefits from or requires measurement data (e.g. (motoric) tasks involving movement or at least moments of immobility of the user), for example. On the other hand, the measurement rate could be reduced to save storage space required, for example.

Various biometric quantities such as skin conductance and/or vital signs, such as body temperature, heart rate or pulse, respiratory rate, and blood pressure, may be measured as well using appropriate sensors. Any of the sensors may attach to the body of the user optionally in skin contact. Implantable sensors may be used. The biometric quantities or specifically responses as well as other quantities/responses may be measured and provided in the measurement data during e.g. VR/AR session or specifically during the therapy involving execution of tasks, for example, and utilized for dynamic determination, optionally adaptation, of the therapeutic program in the light of related objectives and/or security concerns, for example.

In some embodiments, e.g. the amount of pain that the user feels may be determined (estimated) based on the measurements of skin conductance. Skin conductance is altered by the amount of salt/sweat on the skin; this may be associated with the pain that the user feels, as the conductance measures are indicative of ‘physiological arousal’ often elevated in pain. Indeed, skin conductance, breathing rate, blood pressure and heart rate are usually increased when a subject feels pain; any of these may be measured by the arrangement and further reported and/or utilized in dynamic determination of the therapeutic program.

Alternatively or additionally, e.g. camera sensor(s) may be utilized e.g. for automatic facial analysis, which can also be used for detecting pain, fear, lack of those, and/or other condition of the user from facial expressions, for instance. Any applicable software solution may be utilized for the purpose by the arrangement. In addition, a microphone or other sensor can be used to detect breathing, i.e. one of the vital signs. Vital signs, together with movement or position data, can be utilized to estimate the degree of exertion or relaxation among other user status information. These assessments may be made during or immediately after the VR/AR experience or particularly VR/AR based therapeutic session, for example, but additionally or alternatively during other times.

Yet, e.g. the heart rate and/or respiratory rate of the user may be utilized to estimate the level of stress or fear the user perceives (increased level->increased rate).

In various embodiments, the measurement data may be thus utilized to estimate e.g. the immediate effect of ongoing or recent VR/AR experience or specifically, therapy, through monitoring changes in the data while the user is or just was consuming virtual content of e.g. behavior-change and/or user activating domains.

In various embodiments the control system 118 is at least partially if not primarily or solely responsible for controlling the nature and provision of VR/AR content to the user based on e.g. the measurement data provided by the user monitoring equipment 114. The control system 118 is thus configured to dynamically determine the personalized content or generally the therapeutic program including the content based on the measurement data and criteria (logic, threshold values, etc.) available to, such as stored in, the arrangement for deriving proper control measures responsive to the measurement data.

The control system 118 may, depending on the embodiment, comprise computing devices or related elements separate and/or integral with the remaining elements of the arrangement. The devices/elements may be mobile and/or wearable. Typically, at least one processing unit 122 such as a microprocessor, microcontroller, application specific circuit and/or a digital signal processor may be included.

The processing unit 122 may be configured to execute instructions or generally control logic embodied in a form of computer software (program) 126 stored in a memory 128, which may refer to one or more memory chips or memory units separate or integral with the processing unit 122 and/or other elements. Yet, one or more data structures such as database(s) may be established in the memory 128 for utilization by the processing unit 122, storing e.g. virtual content and measurement data. The software 126 may define in addition to general operation or control logic e.g. one or more algorithms/logics for data processing such as evaluation and adaptation of a therapeutic program and/or related virtual content. A computer program product comprising the computer software program 126, i.e. software code means, may be thus provided. The product may be embodied in at least one non-transitory carrier medium such as a memory card, an optical disc or a USB (Universal Serial Bus) stick, for example. The program could be transferred as a signal or combination of signals wiredly or wirelessly from a transmitting element to a receiving element such as the arrangement or specifically, control system 118 thereof.

Item 124 refers to one or more data interfaces (communication interfaces) and control interface/UI (user interface) that may be provided for controlling the arrangement by an operator such as a therapist or other professional and inspecting the data stored in the arrangement regarding e.g. user measurements or user-associated therapeutic program. In some embodiments, the user 201 may be provided with at least limited access to such control measures or features as well. The UI may include local components for data input (e.g. keyboard, touchscreen, mouse, voice input) and output (display, speaker) and/or remote input and output facilities optionally implemented via a web interface, preferably a web browser-based interface, or via dedicated interfacing software. Accordingly, desired, optionally adaptive (as discussed hereinelsewhere), UX may be provided to the stakeholders (users, healthcare professionals, technical operators, etc.) of the arrangement.

The communication interface(s) may refer to one or more wired and/or wireless data interfaces such as proprietary or commonly used wired network (e.g. Ethernet) and/or wireless network (e.g. wireless LAN (WLAN) or cellular) interfaces or adapters for interfacing a number of external devices and systems with the arrangement of the present invention for data input and output purposes, typically including control. The arrangement may even be connected to the Internet for globally enabling easy and widespread communication therewith.

Items 134 may refer to one or more communication connections or particularly communication networks such as the Internet, local area networks, wide area networks, cellular networks, other private or public networks, etc., which enable potentially multiple physically distributed elements of the arrangement and possible external devices or systems communicate with each other.

It is straightforward to contemplate by a skilled person that when an embodiment of the arrangement 114 comprises a plurality of functionally connected devices, any such device or a sub-system of devices may contain any of the afore-discussed elements such a processing unit 122, memory 128, and e.g. communication interface and/or UI 124 of its own for enabling execution of necessary internal functions and mutual/external communication.

In some embodiments the control system 118 comprises a first sub-system 118A, optionally being at least partially integral with the reproduction equipment 114 and/or user monitoring equipment 116, such as VR/AR headset, and further comprises or is at least functionally connected with a second, optionally server-based, sub-system 118B that may be physically remote from or at least physically nonintegral with but indeed at least functionally connected to the first sub-system 118A, optionally via the internet, other communication network and/or generally connection.

The first sub-system 118A may incorporate e.g. local data storage, AI or specifically machine learning algorithm component and/or (further) data analysis component. The second sub-system 118B may contain remote data storage, an AI or specifically machine learning algorithm component and/or a (further) data analysis component. In some embodiments, the predefined algorithms and/or AI/machine learning algorithms (e.g. scorecard type algorithms) executed by the sub-system 118A, if any, may be different, such as simpler, from the ones that sub-system 118B (e.g. reinforcement and/or maximum likelihood type methods) executes. In various embodiments, the control instructions or selected other data provided by remote entities 118B and/or 118C may be configured to generally take precedence over and/or at least used for adapting the local data (determination of therapeutic program, for instance) in the sub-system 118A.

The data stored or processed in the first sub-system 118A may dominantly or solely concern local user(s) only whereas data stored or processed in the second sub-system 118B and/or potential further remote system such as system 118C may at least in some embodiments concern a greater number of users and gathered from several local and/or personal entities such as devices or (sub-)systems 114A, 114B, 118A. In some embodiments, there may not be at least physically separate, mutually remote sub-systems 118A, 118B while the remote system 118C is still implemented. In some embodiments, different approaches for implementing the arrangement can be utilized in parallel. For example, there may be physically mutually integrated as well as mutually remote (but still functionally connected) instances of sub-systems 118A, 118B in the same ecosystem, functionally connected to a common system 118C.

In more detail, the first sub-system 118A may be configured to process measurement data retrieved from the user monitoring equipment 114 and provide at least portion of the processed data to the second sub-system 118B for further processing, storage and/or determination of at least portion of the therapeutic program or related attributes thereat.

The second sub-system 118B may be configured to obtain measurement data and/or data derived therefrom from at least one user monitoring equipment 114 and/or at least one first sub-system 118A, which are potentially associated with (e.g. in the (private) possession of) a single user only at a time, and to process, store and/or determine at least portion of the therapeutic program or related attributes based on the obtained data. Yet, the sub-system 118B may in some embodiments be configured to utilize data regarding several (other) users as well in making the determinations. Such data may be anonymized and obtained from external systems or e.g. system 118C discussed herein.

The first sub-system 118A may be then configured to receive information determining the therapeutic program or related attributes from the second sub-system 118B. The first sub-system 118A may be configured to utilize the data for controlling the VR reproduction equipment 116 to represent virtual content in accordance with the program.

Still, the first sub-system 118A may be configured to autonomously determine (such as adapt) or at least continue executing the therapeutic program and/or control the reproduction equipment to represent virtual content in accordance with the therapeutic program based on the measurement data, responsive to fulfillment of at least one selected condition such as connection failure or connection problem between the first 118A and second 118B sub-systems, or e.g. the failure of second sub-system 118B. The condition may also relate to an adjustable such as user-adjustable or operator/professional-adjustable state of a setting. Yet, the condition may refer to the internal status or capability of the first sub-system 118A to be able to duly execute (accurately and/or rapidly enough, for instance) the necessary activities for determining the program or continuing executing it autonomously, which may be monitored by the first-system 118A itself.

Any of the entities 114, 116, 118 or their sub-systems or component devices may be portable (also) in terms of operating power, i.e. they may be capable of operating by means of included preferably rechargeable battery and/or powered wiredly or wirelessly by an external power source. E.g. the reproduction equipment 116 may include internally or wirelessly powered devices such as a headset or other wearable projection device. The same also applies to equipment 114 such as optionally hand(-held) controller(s).

Item 118C refers to a remote treatment management system or platform, which may be utilized, among other options, to facilitate the treatment of the user(s) e.g. between different health care professionals and/or other parties by offering data collection and integration as well as interaction and/or control facilities therebetween. The entity 118C, optionally at least partially established by a number of servers, may be configured to provide data such as instructions/control data and/or statistical data towards and/or obtain data such as status/measurement data from at least one but typically a plurality of users or their local instances of the arrangements and/or related equipment 114, 114B, 116, other (terminal) devices, and/or sub-systems 118A, 118B when applicable.

In some embodiments the system 118C may be included in the overall arrangement 118 e.g. as or in a sub-system whereas in some others, it may be considered to constitute an external remote system functionally (communications-wise) connected with the arrangement 118. In any case, when the arrangement is just generally mentioned below to execute some higher level processing or storage action regarding e.g. several users or not at least requiring physical vicinity to any specific user, a skilled person shall realize the fact that also the entity 118C could be configured to execute the action notwithstanding the fact whether it is considered as the part of the arrangement or just a functionally connected entity.

In more detail, the remote system 118C may be configured to provide access to data obtained from or established in any of entities 114, 116, 118A, 118B, 118C for system or user (progression/performance in terms of the therapeutic program, status, etc.) monitoring purposes to various parties such as therapists or other healthcare or technology professionals, for example. As mentioned hereinbefore, the system 118C may obtain the data e.g. from at least part of the equipment 114B via routes not involving other elements of the arrangement 118, with reference to alternative communication channels. For example, the equipment 114B may contain terminal devices such as a personal computer or mobile terminal/smartphone that can be instructed, optionally by client software running thereat, to address data directly to the entity 118C via the internet, for instance. The aforementioned parties may in turn use the system 118C locally or remotely e.g. via their terminal devices (e.g. computer devices or mobile terminals such as smartphones). The system 118C may provide access for e.g. control, monitoring, advisory, data input, data output and/or support purposes to any of the parties to render them capable of communicating with one or multiple user(s) and/or associated one or more arrangement(s) 118 or sub-systems 118A via the at least conceptually centralized system 118C. Accordingly, data transfer between systems 118C and any of 118, 118A, 118B may be bi-directional.

The system 118C may be configured to provide data such as aggregate data, optionally statistics concerning one or several users, potentially in anonymized format and/or control instructions obtained from or determined based on data obtained from the connected parties (health care professionals, users, etc.) via their terminals/systems to the target elements of the arrangement 118 (e.g. sub-systems 118A and/or 118B) for various purposes such as therapeutic program determination including selection or adaptation, for example.

Yet, communication facilities (e.g. voice, video or messaging link(s) or platform(s)) may be provided, e.g. at least partially via the system 118C and/or other discussed entities, for real-time and/or non-real-time communication between the stakeholders such as healthcare professionals and users, or between several users of the arrangement.

In various embodiments, the system 118C may be configured to store data such as data received from the (remaining part of the) arrangement and/or from other systems or devices such as external systems/devices of healthcare professionals either as is and/or in processed form. Yet, the system 118C may indeed be configured to process the received data and determine, for instance, selected statistics or other aggregate indicators therefrom, as well as control instructions or other data for use by the (remaining) arrangement e.g. in determining the therapeutic program such as adaptation of session or module parameters or associated tasks. The received data and/or indicators derived based thereon may be forwarded to external systems/devices for local use thereat, optionally storage, analysis, inspection, etc. The data/indicators may imply, among other options, the status or characteristics of a user, related measurement data (e.g. sensor data, real-life data, subjective data such as afore-discussed diary or questionnaire data, etc.) and/or their progression or performance in terms of the therapeutic program (performance in conducting one or more tasks, current stage in the therapeutic program, etc.), for instance.

Virtual support or peer support involving remote communication with other parties has been found advantageous in the context of the present invention as e.g. the users, which may utilize their own instances or at least portions of the arrangement in “isolation” (at home or summer house, on a trip, etc.), may still appreciate and benefit from changing thoughts with and getting advice from others such as healthcare professionals or other users.

Yet, as mentioned hereinbefore aspects of gamification may be extended to peer communication with reference to common scoreboards (may be therapy program, module/session and/or task related, for example) and possible other comparative if not competitive data.

Instead of or in addition to visual or graphical such as textual, video and/or avatar-based communication, voice/audio communication may be enabled between the parties by the communication features of the arrangement.

Accordingly, the arrangement may be configured to provide real-time and/or nonreal-time communication channel or platform between users and/or between a user and at least one other human party, optionally a health care professional or e.g. friend, relative or other person willing to support the user in their therapy, preferably in a virtual environment and space wherein one or more of the communicating parties are represented advantageously graphically, optionally by avatars.

Optionally, any of the communication features provided utilizes automated language translation provided in the arrangement or connected system to facilitate communication between the concerned parties. The arrangement may, at least in terms of some of its features (including e.g. virtual therapist) additionally support multiple languages and preferably select the language for interaction with or presentation of information to the user according to e.g. user input or other control input regarding the same.

Thus in some embodiments, also from the standpoint of e.g. gamification, offering visibility if not even actual communication facilities between several users may turn out advantageous, because it may add to the motivation of the users to continue with the therapeutic program and/or execute the associated tasks. For example, performance data (solving time, scores, etc. regarding e.g. tasks, sessions/modules or programs) of a plurality of users may be shared therebetween via the arrangement (and e.g. system 118C if implemented and not considered at least essential part of the arrangement itself) optionally in anonymized or pseudo-anonymized format, based on e.g. user-selectable user identifiers disclosed.

In some embodiments, a virtual communication party may be created to interact with a user for advisory, support or other purposes. In more detail, the arrangement may be configured to represent, visibly and/or audibly, a computer-generated, preferably artificial intelligence based, virtual therapist or other artificial person with a characteristic visual appearance, optionally a graphic figure such as an avatar, and/or voice to the user e.g. via the reproduction equipment, and further configured to provide the user with instructions, support or feedback optionally regarding the use of the arrangement or the therapeutic program via the virtual therapist/person.

For example, the arrangement and e.g. the virtual therapist feature therein may be configured to monitor the status or performance of the user, or the progression of the user, in relation to the assigned therapeutic program or any of the associated tasks, for example, and based on the rules coded in the operating logic (may be predetermined and/or based on AI) of the feature, trigger actions such as communication activities responsive to the monitored data.

For example, if the user appears to be struggling with the therapeutic program according to the criteria utilized by the logic, the virtual entity may be configured to issue supportive and encouraging statements towards the user (statements may be selected from a plurality of options associated with different user statuses/performances).

An existing AI engine may be selected and configured for use here, or a proprietary one may be alternatively utilized, in cases wherein e.g. a virtual therapist is desired to be at least partially implemented through AI.

Also generally in various embodiments the arrangement may be configured to utilize an AI or specifically machine learning algorithm for selected purposes, e.g., for (dynamically) determining (adapting, changing, etc.) the therapeutic program.

For example, the AI/machine learning algorithm may associate e.g. sensor-based (objective) measurement data and/or (user-created) subjective data, or data derived therefrom, with selection or adaptation of the therapeutic program or at least with interim result to be utilized in determining (adapting, for instance) the therapeutic program optionally by a different type of logic or algorithm. The interim result could refer to determining e.g. user-related status information or other characteristic data regarding the user or their progression or performance in conducting a task, for example. The adopted solution such as AI based solution shall still preferably operate within defined safety constraints in terms of e.g. tasks assigned to the user. Safety issues have been discussed in more detail hereinelsewhere.

In various preferred embodiments, utilization of algorithms and logics falling under, for instance, AI, or specifically machine learning, for providing a virtual therapist, other form of user guidance, performance evaluation, therapeutic program determination/adaptation and/or some other feature may involve a plurality of mutually compatible and also jointly applicable approaches, examples of which are reviewed below.

For example, the performance of an individual user and how they interact with the arrangement may be inspected by the arrangement, whereupon related measures may be taken. Among other options, it may be determined e.g. from the usage statistics that the particular user avoids or over-engages with one or more elements such as therapeutic programs or tasks (which both may define or be included in a number of training or exercise modules or sessions indicated to the user for participation) or e.g. interactions (with virtual or real health care professional such as a therapist, and/or with other user(s)) implemented by the arrangement.

In response to detecting a behavior of interest (e.g. deviation from a planned activity in over-use or under-use (or essentially non-use) of an activity) according to a selected criterion, the arrangement may be configured to identify the deviation, and determine and execute a response. The arrangement may be thus configured to respond empathically and/or motivate behavior change, based upon the therapeutic principles and practices programmed/configured to the arrangement.

For example, suitable feedback may be given to the user in a safe zone type space (may include e.g. instruction, relaxation and/or other content from behavior-change content domain) of the VR/AR environment produced, e.g. by an avatar. Encouragement to complete the planned tasks may include visual/graphical and/or vocal/audio parts. The user may be also conditionally offered a new experience provided that the feared/disliked task is completed first. Based on e.g. user status or statistics, experience such as a task or other experience (e.g. a reward experience that could also be passive by nature requiring no substantial user activity) may be deemed potentially interesting from among all available options and selected for the offer, and/or the selection may be based on analyzing which kind of experience such as task would benefit the user from the standpoint of their medical condition and therapeutic program most.

The user may be thus actively guided and encouraged by, for example, providing the user with content intended to effect such goals, to successfully finish all of the planned tasks of the therapeutic program.

As another example, by the monitoring equipment 114 including e.g. sensors, changes in vital signs of the user may be detected in the biometric data, which may be deemed sensations of pain according to the criterion used in assessing the data (e.g. values going beyond set thresholds or leaving below them) as deliberated hereinbefore. The arrangement may be configured to avoid directing the user into such situations (avoid representing the user virtual content of type and/or duration, e.g. in the form of a task to be conducted, which triggers the undesired effect) regularly or at least too often according to the utilized criterion, but e.g. still occasionally if being part of the therapeutic program and associated task assigned to the user to deal with their medical condition.

In a further example, several users are jointly analyzed optionally by AI or specifically machine learning and/or other operation logic. Such analysis and processing tasks involving data regarding the users may be in some embodiments specifically conducted in an entity such as system 118C, or in some embodiments alternatively or additionally sub-system 118B (whenever implemented), functionally connected to one or more local (instances of) systems 118A and/or equipment 114, 116 of the actual users.

In more detail, the arrangement may be configured to determine a number of selected descriptors such as mean, median and/or outliers regarding the users participating e.g. in the same or similar therapeutic program, conducting same or similar tasks, etc. This could be at least partially done by entities that are remote from each user's location, such as in some embodiments at least sub-system 118B and/or system 118C. The determined data could be utilized to instruct the control system 118, 118A closer to/associated with each user for executing the VR/AR experience. For example, a therapeutic program could be dynamically determined in terms of associated temporal issues such as scheduling of tasks or sessions based on satisfactory results-bringing scheduling from the standpoint of the majority of users. Likewise, generally less optimal scheduling of the therapeutic program could be avoided.

Still as a further example, if it is determined a particular user is, based on the statistical data gathered by the monitoring equipment 114 associated with the user, acting sub-optimally or abnormally, proceeding e.g. hastily through content without paying enough attention to it (spends too little time inspecting it and/or reacts to it too quickly), the arrangement may be configured to approach or specifically notify the user about the matter. For example, if the user is detected to answer questions provided by the arrangement very rapidly or the answers statistically follow some statistically unusual pattern (e.g. from multiple options the user always selects the answer that has similar spatial positioning, e.g. topmost answer, or the answers just deviate from mean/median ones of a greater group of users sufficiently), the arrangement may be configured to re-introduce the questions to the user and/or execute some other verification or notification action.

To conclude the review of FIGS. 1-2 and discussion around more or less general aspects of various embodiments of the arrangement, and as already mentioned to some extent above, in some embodiments at least part of the arrangement may be personal to a user or limited group of users, whereas the remaining part or at least the external systems/devices coupled to the arrangement may be utilized by a greater number of users and/or entities. For example, equipment 114 and 116 may be personal and/or be in the possession of a single user at a time, which may further apply also to the control system 118 or at least the first sub-system 118A thereof in case the control system 118 is a multi-device system with physically separate and remote sub-systems 118A, 118B and/or system 118C.

Yet, at least part of the devices of the arrangement, such as one or more devices of sub-system 118A, and particularly sub-system 118B and/or system 118C, may reside in a server (e.g. blade) and/or in a cloud computing environment and be dynamically allocable therefrom if a need for extra resources in terms of computational or storage power arises, for instance.

Hereinbefore, certain virtual spaces, zones, (sub-)environments or modes of the overall virtual environment produced, such as a ‘safe mode’ (or ‘personal space mode’) and an ‘activity space mode’ have already been briefly discussed. The former may be used as the safe environment for providing e.g. relaxation, virtual therapeutic advice and/or other type of behavior-change content and therapy to the user, and/or for managing the VR treatment (otherwise) whereas the latter may be used to execute the therapeutic treatment relying upon e.g. principles of VR game design, level design and algorithms, thus involving gamification and incentivization. Desired behavioral responses may be obtained from the users without cognitive decision-making. This may be achieved by immersion and content design which preferably targets person's internal and intuitive decision making and incentivization process.

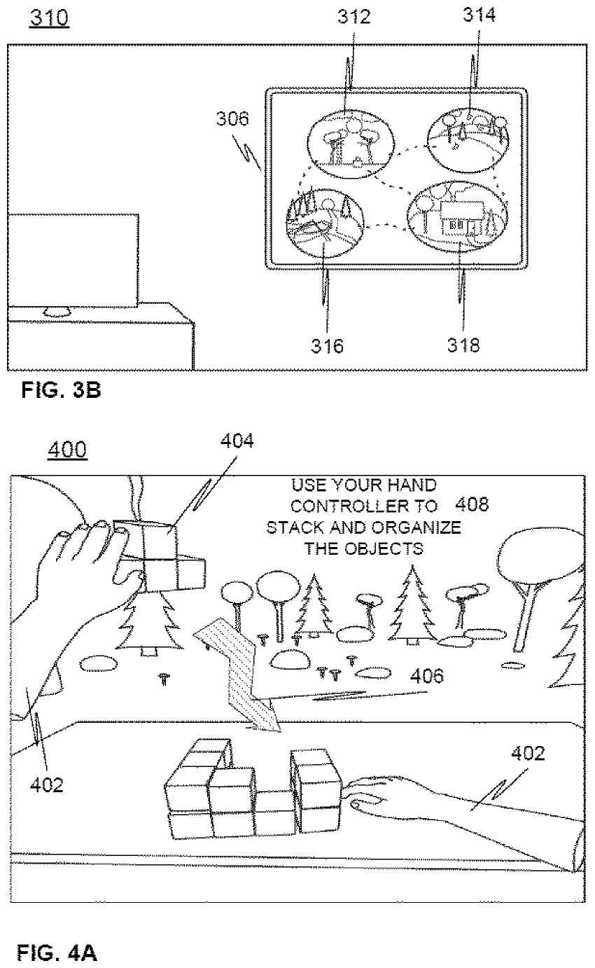

FIG. 3A illustrates, at 300, a first person (screenshot type) view incorporating behavior-change such as relaxation content that could be provided to the user via the reproduction equipment of an embodiment of the arrangement of the present invention, while FIG. 3B illustrates, at 310, a more focused first person view from alternative position in the virtual space/virtual environment depicted in FIG. 3A.

FIG. 3A may depict the safe or home space, zone, or mode wherein the user may preferably stay and act (move, address items, etc.) in a relaxed fashion while feeling comfortable. Accordingly, the reproduced virtual content including a plurality of virtual items 302 (e.g. window (view)), 304 (e.g. furniture), 306 (e.g. a painting, poster, or a notice board) may include content of behavior-change, and more specifically, relaxation type, for instance.

The content reproduced in this or other virtual spaces may be adapted or personalized for the user based on related data input by the user (e.g. questionnaire, photographs regarding their home or (other) pleasant, secure places) and/or an operator/healthcare professional, and/or on various contextual attributes (e.g. current geographical location, user demographics (age, gender, etc.), time (of day)), which may take place automatically, e.g. by the arrangement relying upon predefined parsing or other data analysis logic applied, and/or manually by healthcare and/or technical professionals, for example. Yet, the content may additionally or alternatively be adapted based on the therapeutic program/medical condition associated with the user, characteristics or status of the user, and the user's performance in conducting the assigned tasks, for instance, as contemplated in more detail hereinelsewhere.

Various measurement data including volitional control input by the user may be obtained e.g. by a number of appropriate sensors that included in the user monitoring equipment 114 as also discussed hereinbefore. For example, detected rotational or translational motion of the user or their body part such as head or limb in the physical world may be converted into corresponding, similar or different motion of the user in the virtual world by the arrangement in accordance with related conversion rules and logic applied by the arrangement, which may optionally be personalized to adapt to e.g. physical dimensions of each user and/or physical (real-world) space where the user accesses the content into account.

In the safe space depicted in FIG. 3A the user may be thus provided with one or more options to move to other venues in the virtual environment through the use of applicable control features supplied with the arrangement such as hand controller(s) or a sensor-provided headset.

Preferably, in various embodiments, entry into other virtual spaces incorporating e.g. other domains of virtual content such as user-activating content or other types of generally behavior-change type content such as fear confrontation content may be thus triggered responsive to volitional user input for the same. It could be additionally or alternatively configured to take place also automatically or at least more user independently such as in a timed fashion, externally triggered e.g. by healthcare professional, (pseudo-)randomly or based on measurement data not indicating explicit user instructions to move into the other space but instead indicating e.g. user status, which in the light of the therapeutic program and related objectives is configured to trigger transition between the virtual spaces.

In FIG. 3B, the user is shown standing closer to the item 306 in the same virtual space. The user has thus moved in the virtual space by giving corresponding control instructions to the arrangement via a controller device of the user monitoring equipment, for example. The item 306 may be configured to represent a visually readily identifiable access mechanism or destination towards the other virtual space(s). It 306 may be configured to graphically indicate, e.g. by descriptive figure(s), symbol(s) and/or text(s), optionally supported by reproduction of descriptive audio signal, the nature of the target virtual space(s) and/or content (e.g. content domain, tasks, therapy module/session ID or other information), which may be entered by a predefined control input from the user indicative of the selection of a desired target space/content. Again, the control features associated with the arrangement (e.g. a button of a hand controller) may be used for the purpose.

In the shown view and scenario, the item 306 exhibits four identifiable elements 312, 314, 316, 318, each of which may lead to different virtual space, therapeutic module/session and/or content domain in the virtual environment.

The nature and number of accessible virtual spaces or content domains may dynamically vary even within a common virtual environment. If they represent e.g. different exercises or training sessions involving tasks to be conducted, only the ones that are deemed suitable to the user in the current stage of a therapeutic program may be indicated to the user or granted access with.

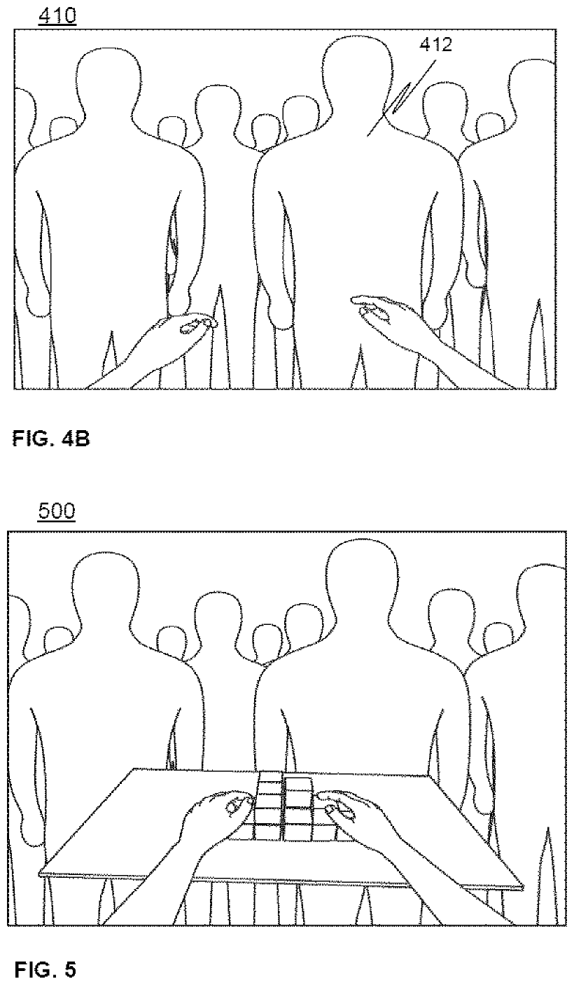

FIG. 4A illustrates, at 400, a view such as a display (screenshot) view rendering essentially user-activating virtual content that may be provided to the user via the reproduction equipment of the arrangement. Using the terminology of modes as relied upon hereinbefore, FIG. 4A may be considered to depict the ‘activity space mode’.

In various embodiments, the virtual content indicative of the series of the tasks to be conducted may comprise and visualize one or more virtual target objects reaching, manipulation or other addressing of which in the virtual environment or virtual part of a virtually augmented environment by associated therapeutic activity such as physical movement in the physical world enables to achieve the tasks.

Yet, at least one virtual target object may define e.g. a geometric shape, optionally a tetromino, polyomino, tetracube, polycube or alike, to be preferably acted upon and, for example, manipulated by rotation, translational movement, introduction, removal, resizing or reshaping in the virtual environment or virtual part of the virtually augmented environment, optionally through conducting similar activity in real-life by the user as indicated by the measurement data.

In the scenario of FIG. 4A, tetromino type geometric virtual target objects 404 are presented to the user e.g. one or several at a time. The user may then pick and manipulate the objects 404 by rotation and translational movement by using their virtual hands 402 the position of which in the virtual space may follow their positioning in the physical (real) world in front or generally field of view of the user. The activity is set to take place in a selected virtual space or environment such as in the visualized case, a virtual forest rendered in the background. In some embodiments, certain tasks may be associated with a selected or selected type or class of virtual space in addition to e.g. virtual objects to be manipulated or acted upon.

In various preferred embodiments of the present invention, the user-activating virtual content indicative e.g. of a series of tasks (through visualization thereof and/or of the associated virtual content items such as the above tetrominoes, for instance) to be conducted further advantageously visualizes the nature, outcome, progress, goal and/or execution of the associated therapeutic behavior, such as physical activity, to be performed by the user in the real world to advance the execution of one or more of the tasks. Such visualization may be implemented utilizing e.g. one or more alphanumeric characters (optionally defining instructional text), symbols, pictures, and/or animations in the virtual or virtually augmented environment.

In some embodiments, the virtual content indicative of the series of the tasks to be conducted comprises audio data such as spoken (human recorded or synthesized speech), melodic or otherwise preferably descriptive audio instructions.

In the scenario of FIG. 4A, the virtual content includes clues 406 (arrow symbol), 408 (text) indicative of the therapeutic behavior, in this case at least hand movement, to be performed by the user in real world to advance the virtual task(s) of arranging and/or stacking the tetrominoes 404, for example, on a surface such as a table in the virtual environment and active virtual space thereof. Yet, any of the clues 406, 408 could in some embodiments represent example of guidance provided to the user during therapy by a virtual therapist feature implemented in the arrangement.

In the shown example, the clues 406, 408 in addition to reflecting the desired therapeutic activity in the real world actually also instruct the user in terms of executing the tasks in the virtual space (indicate the tasks in the virtual environment), which may often be a preferred implementation when e.g. movement is expected from the user. In other words, to make execution of a task more understandable or easier, various aspects such as directions in the physical world and virtual environment may be configured to substantially match.

FIG. 4B illustrates, at 410, a further (screenshot) view of a virtual space or environment comprising fear confrontation type behavior-change content to treat e.g. selected phobias.

The user may suffer from a fear of open spaces, crowds and/or a reluctance to engage in busy environments because of e.g. a lack of control over anxiety or avoidance of pain. Accordingly, the arrangement has been configured to virtually situate the user in or close to a fear-causing virtual object or feature such as a crowd 412, with reference to principles of exposure therapy, for instance. The user may be enabled move translationally or rotate, or any of such motion may be disabled. The user may be also given tasks in the virtual environment, which may require, to succeed, certain therapeutic behavior or specifically e.g. movement, immobility/lack of movement (just consuming/perceiving the (assigned amount and/or type of) content in extreme case, for example), and/or other response from the user, for example. Thus not all tasks issued by the arrangement have to be motion-related or at least dominantly of user-activating type in terms of substantial physical activity. The user may be provided with encouraging content such as messages optionally responsive to the receipt of measurement data indicative of e.g. increased fear or stress.

The measurement data obtained in the scenario of FIG. 4B and other situations involving the provision of e.g. behavior-change content to the user (e.g. the scenarios of FIGS. 3A-3B) may indeed contain, among other options, biometric data, movement data and/or other data indicative of the user's status, characteristics, and/or performance in conducting a task, for example. Yet, more subjective data provided by e.g. the user and/or therapist/healthcare professional (evaluating/monitoring the user on the spot or remotely) may be obtained. The data may be utilized for dynamically determining, optionally adapting, the therapeutic program and/or (at least behavior-change type) virtual content provided via the safe space or other virtual space in the virtual environment so that the objective associated with space or content is achieved.

For example, quantities such as heart rate, respiratory rate, blood pressure or skin conductance, which may be utilized to estimate the level of relaxation or e.g. fear, may be measured and used to control the virtual content provided to facilitate the user reaching or getting closer to a desired level in this respect from the standpoint of the objective of the therapeutic program. E.g. a peaceful and/or calming scene or view (e.g. ocean or sunset view as a concrete example) could be visualized to the user when further relaxation or reduction of fear is desired based on the measurement data indicative of e.g. undesirable high values in respect of any of the above quantities according to selected criteria. In contrast, as described above rather different if not opposite type of content such as fear-causing content could be provided if the user is to be confronted with a source of fear or activated otherwise.

FIG. 5 illustrates, at 500, still a further view of a particular virtual environment or particular space within the environment combining different domains of virtual content (in this example, essentially simultaneously but also alternating sequence could be considered in some other embodiments; in other words, generally a virtual space could be associated with a single domain or multiple different domains of virtual content), which is also a possible scenario in various embodiments of the present arrangement. For example, behavior-change content and user-activating content such as gamified and/or incentivized content may be utilized simultaneously. With one content, the disturbing effect of the other may be reduced, for example.

Generally, the user may be thus distracted from perceiving the actual symptom, related fear or content (associated with e.g. certain fear or pain) by certain content the user deems inspiring such as user-activating content.

With reference to various virtual content provided to the users by the arrangement, including behavior-change content as well as e.g., user-activation content, the following guidelines are disclosed for at least selective application in creating such content for treating various medical conditions such as chronic pain:
a) relationship maintenance (developing a trusted relationship between the user and the mentor such as healthcare professional/therapist or other agents with the space): any instruction for behavior change has the opportunity to be ineffective or even harmful. A basic principle of behavioral instruction is to establish an alliance between the therapist/instructor/mentor and the user considering change.

There are many strategies for establishing alliance in face-to-face delivery, but in the context of the present invention novel features can be created for establishing and maintaining a relationship with e.g. a virtual therapist or mentor that may be AI based or otherwise programmed and/or algorithmically created, for instance. In order for the arrangement or specifically e.g. the virtual therapist/mentor implemented therewith to successfully encourage, instruct, require, or advise on behavioral change, selected textual, graphical (e.g. avatar's expressions), sound-based or other forms of expression may be provided to the user to establish trust and empathy. Yet, e.g. the operation logic supervising the selection and/or execution of tasks, which may further be based on AI, can be configured to determine the tasks to be maximally or suitably challenging in which failure is not avoided but is minimized and paced, supplemented with setback planning to learn from failure and positive reinforcement of planned behavior.

It is advantageous to personalize the VR/AR experience also from this standpoint by suitable choice of mentor features (visual style, messaging/communication style, voice, etc.), for example. Yet, specific content to manage relationship fracture in which trust or belief is challenged by experience may be utilized.
b) embodied reactivity (acting in the physical space, using e.g. movements in all quadrants, reaching, stretching, and/or manipulating virtual objects): to reduce e.g. the micro-avoidance of painful movement (e.g., guarding, rubbing, holding) tasks to produce movements in e.g. all four quadrants of peripersonal space may be created, encouraging exploration with both hands and/or involving movement, preferably critically above the head and below the waist, in a paced self-determined manner.
c) courageous engagement: to counter e.g. the fear of pain on movement, the engagement (sometimes called approach behavior) with the feared movement is encouraged, which is recognized to involve a person risk, courage, bravery and determination, and at times tenacity. Understanding the exact driver of avoidance, whether a fear of harm, of negative social judgement, of failure, of identity challenge, etc. is an important first part, as is tailoring content to the specific context of the pattern of avoidance behavior requiring confrontation and exploration of the consequences of safe confrontation. Again, using e.g. AI, text, metaphor, planned activity, and/or exposure in virtual environments the engagement with the feared movements may be instructed, and then provoking insight and learning about the possibilities of movement despite the possible pain in which the feared consequences do not follow, however.
d) mastery (increasing problem-solving skills and confidence): e.g. chronic pain creates multiple and repeated failure experiences in all aspects of emotional, cognitive, behavioral, and relational tasks. Repeated failure creates a helplessness and in some a hopelessness. The solution in accordance with the present invention may be configured to provide opportunities for success in multiple task environments which can be positively reinforced, success in planned behavior leading to longer term improvement can be visualized and presented to reinforce paced engagement, cognitive and social problem solving can be attempted, practiced and reinforced. Existing protocols to transfer individuals from being externally reinforced to become self-reinforcing may be utilized.

With reference to various embodiments of initially adapting or basically calibrating the arrangement for a new user or assessing the user's status or desired characteristics for determining suitable therapeutic program or related tasks, the following remarks and practical examples are given for measuring and analyzing the user.

A baseline status can be determined e.g. on the first experience of the arrangement, which may be optionally implemented through a dedicated calibration or initial deployment mode, or the status may be determined during e.g. first ordinary VR/AR experience involving e.g. user-activating and/or other type of virtual content. Subsequent exposures can then show progression.

For example, a user suffering from pain such as low back pain may have restricted movement during the VR/AR experience. A volume curve as defined e.g. by the Cartesian x,y,z coordinates, or linear movement along e.g. the y coordinate (Height) for the headset and the hands may be used to indicate the associated range of movement.

Responsive to the (initial) measurements, the arrangement may be then configured to dynamically determine a therapeutic program wherein e.g. tasks assigned to the user may involve target movements about e.g. 70-100% of which are at least at first, e.g. on day one and/or during a number of first exercises or sessions, maintained within the initial/natural motion range of the subject.

In another example, a user may show limited movement and does not enter e.g. crowded areas based on data such as sensor (e.g. motion and/or positioning) data and/or subjective data such as questionnaires. This may indicate e.g. kinesiophobia, or generally pattern of feared harm or pain on movement, and the related status analysis regarding the user may involve utilization of Tampa Scale, for instance.

In a further example, a user may be detected to use their smartphone in the beginning of the day, but the devices may record physical movement in the evening. This may indicate morning stiffness and e.g. medicines taking effect later in the day.

At least three data input categories for (machine learning and other) algorithms utilized by the arrangement may be generally identified:
1) Data gathered during a VR/AR session including therapeutic session
2) Data gathered over the VR/AR sessions as a process over time
3) Other physical/real world data collection (e.g. aforesaid activity, sleep, PROM questionnaire and other data) as a process over time

VR/AR reproduction equipment and user monitoring equipment, such as a VR headset and handheld (control) devices (e.g. one in either or both hands), may be equipped with inertial sensors such as accelerometer and/or gyroscopic sensors. Data collected from the available sensors may be utilized to determine the three-dimensional location/position (p) of the concerned sensors in a certain time point (t). As the user starts the exercise (t=0, p=X0, Y0, Z0) the starting position for a sensor may be measured and considered as the origo or generally zero/reference point or origin. When a therapeutic session begins, the user starts moving his/her limbs and head according to the given tasks and personal capabilities. Thus, each sensor will move in the three-dimensional space and that movement can be monitored by the monitoring equipment. The movement can be expressed by using e.g. a 3D-vector over time which is a mathematical expression for the distance of the sensor from the origo position of each sensor. Thus a function of 3D-vector can be collected over time for each sensor and selected one or more attributes calculated from this function, e.g. max, mean, average, standard deviation (SD) and RSD (relative SD) for the 3D vector (including the amplitude of the length of the vector) of each sensor, which can be then expected to increase during successful therapy if the problem was in the movements of the user, such as limited range of motion, in the first place, or related factors such as fear of movement.

Yet, based on such data from several sensors so-called relative 3D vectors may be calculated, which are basically expressing the distance between selected objects, such as between right hand and left hand, right hand and head and/or left hand and head. Again, several values for associated distances may be determined, e.g. max, mean, average, SD and RSD for the relative 3D vector (including the amplitude of the length of the vector). Further, these values may be expected to increase when the therapeutic program advances.

With reference to the aforementioned baseline status, when using the VR/AR software for the first time, the user's range of movements can be tested by user moving hands/head on all directions as much as comfortably possible. This can be considered as the baseline/initial calibration for subsequent movements.

In the light of the foregoing, in various embodiments the arrangement may be configured to obtain an indication of the medical condition and/or selected anthropometric, musculoskeletal or physiological characteristics of the user, such as range of motion, optionally through utilization of the user monitoring equipment and measurement data acquired therewith, and to preferably (dynamically) determine the therapeutic program based thereon.

Still, in various embodiments the arrangement may be configured to compare first measurement data or data derived therefrom relating to a first body part with second measurement data or data derived therefrom relating to a second body part and/or a reference point, and based on the comparison result preferably additionally to determine an indication of the medical condition and/or selected anthropometric, musculoskeletal, physiological or other characteristics of the user, optionally comprising an indication of flexibility or range of motion.

The comparison may involve subtractive comparison, such as calculation of mathematical difference and optionally involving vector calculus as contemplated hereinbefore.

In various embodiments, the first measurement data or data derived therefrom may concern head, trunk, or first limb of the user, optionally upper limb or a portion such as shoulder, arm, upper arm, forearm, and/or hand thereof, and the second measurement data or data derived therefrom may concern e.g. at least second limb, optionally upper limb or a portion such as shoulder, arm, upper arm, forearm, and/or hand thereof, of the user.

In various embodiments, a selected characteristic such as range of motion may be determined for at least two anatomically or functionally corresponding body parts such as both hands or both legs of the user, preferably relative to e.g. head or other origo/reference point as discussed hereinbefore. Differences in the measured characteristics between the body parts may be utilized by the arrangement to obtain the indication of medical condition, its severity or other characteristics regarding any of the parts.

For example, the one with reduced range of motion or other measured incapacity may be deemed injured or requiring therapy. For the therapy, the capability of the other body part may be used as the objective for the therapy, or for determining the objective (e.g. proportion thereof may be selected as the objective) together with other possible information characterizing the user as discussed hereinelsewhere.

In various embodiments, the compared data such as the first and second measurement data comprise motion data, optionally provided by at least one inertial sensor such as accelerometer of the user monitoring equipment as contemplated above.

With reference to the personalized therapeutic programs and their dynamic determination (adaptation, for example), one possible embodiment of a therapeutic program hosted (preferably stored and maintained) and provided by the arrangement comprises or is embodied in a data collection such as a data structure of digital content that may be arranged into a desired sequence where content from different content domains may alternate and/or be simultaneously reproduced thus potentially overlapping both temporally and spatially (e.g. shown superposed via a display).

In addition to or instead of directly arranging (selecting or scheduling, etc.) virtual content involving e.g. behavior-change content and/or user-activating content items into a therapeutic program, the program may be defined by a number of intermediate elements called e.g. therapeutic sessions (or modules), each potentially associated with desired virtual content indicative of e.g. the series of tasks to be executed during the concerned session so that the objective of the program such as improved range of motion or reduced phobia can be controllably reached. The sessions may be thus scheduled to establish the overall program and have their own focus areas and objectives in terms of e.g. included virtual content.

In some embodiments, the concept of modules could be adopted with similar content and/or objectives as mentioned above in addition to the concept of sessions. A therapeutic module could be then completed during and as split into a number of VR/AR therapeutic sessions, which in turn could be predefined e.g., in terms of content duration and/or number thereof by the arrangement or be at least partially dynamically user-selectable in that sense based on their personal preferences, such as time available for a session or current state of alertness of the user.

Thereby, in some embodiments a module could be defined as an entity that can or must be completed during a number of, or specifically, a plurality of therapeutic sessions, whereas in the other embodiments there does not have to be a separation between the concepts of a module and a session.

In various embodiments of the present invention, a personalized therapeutic program determined for the user may define, comprise, and/or link to at least one element selected from the group consisting of:
medical condition to be treated,
objective(s) to be attained,
virtual content domain(s) preferably including the ones applied in the program,
virtual content (items) including e.g. virtual target objects to be visualized to and interacted with by the user (and e.g. related attributes such as applicable manipulation methods and/or behavior), and/or the ones being e.g. static and/or in the background,
tasks associated with the virtual content,
timing information regarding e.g. the therapeutic program and/or constituent tasks, series of tasks, session(s), module(s) and/or related objective(s), and/or related recovery periods,
user status and/or performance evaluation criteria, which may optionally include e.g. evaluation logic and/or evaluation values such as threshold values utilized by the logic by comparing them, for example, with the measurement data (criteria may be for physical activity and/or other therapeutic activity/behavior required to be satisfactorily executed in real world to advance and thus linked with the task or series of tasks in the virtual environment, where the behavior is typically in favor of the overall objective(s) of the therapeutic program), the criteria may concern e.g. a task or series of tasks, a session, and/or a module, and
(therapeutic) session or module information such as content thereof (e.g. tasks to be conducted, virtual content items, required therapeutic activity, sequence and pacing of included task, etc.).

The timing information mentioned above may refer to e.g. scheduling, pacing and/or duration data of any of the listed items including or omitting possible idle or intermediate periods (periods not involving therapy, other planned activity or even use of the arrangement).

Dynamic determination of the personalized therapeutic program may comprise configuring/adapting any of the afore-listed elements or their components, for example.

Yet, dynamic determination of the personalized therapeutic program may generally comprise initial determination and/or subsequent adaptation any of its elements based on e.g. measurement data or explicit control input from e.g. responsible healthcare professional/therapist or the user themselves.

One or more of the above elements associated with the therapeutic program may be personalized to a target user or a group of users, for instance, and thus render the whole program respectively personalized to a selected extent.

Hereinbefore, determining baseline for the user or calibrating the arrangement for the user has already been discussed. Based on the baseline data and/or other status or characteristic information available regarding the user (such as indication of symptom(s) and/or actual medical condition of the user, and/or height/weight/age/gender type information, for example), a personalized therapeutic objective such as a target movement range and/or a psychological target may be determined.

Generally, e.g. at least one database or other data structure(s) accessible by the arrangement and/or operation/control logic, optionally incorporating aspects of AI such as machine learning, may be used in linking various available data characterizing the user together e.g. into an objective for a therapeutic program including e.g. range of motion and/or fear/phobia related targets.

Hereinbefore it has been already described how different measurement data may be obtained and e.g. mutually compared optionally by difference calculation to derive interesting indications of e.g. the range of motion of the user. In some embodiments, the arrangement may be configured to dynamically determine the therapeutic program based on the comparison result (by further comparing the comparison result with selected threshold value(s), for example) optionally comprising selecting or configuring (adapting, for example) one or more tasks of the series of tasks and/or the extent of associated therapeutic behavior needed to advance the tasks.

In various embodiments, the dynamic determination comprises adapting the therapeutic program such as the included virtual content of any of the domains, optionally at least the user-activating virtual content, based on the user's performance in conducting the series of associated tasks in the light of the measurement data and e.g. applicable performance evaluation criterion or criteria describing real world behavior such as physical activity required for the task(s) to proceed in the virtual environment. For instance, in the context of physical movement related target behavior, the criterion could define e.g., by at least one threshold value, sufficient range of motion of a body part to successfully proceed with or finish the execution of the task(s). In the context of treating e.g. phobia, the criterion could correspondingly define e.g. maximum level of fear allowed to successfully execute the task(s), which may incorporate exposure therapy, for instance.

In various embodiments, the dynamic determination may comprise at least one action element such as adaptation element selected from the group consisting of:
selecting a task from a plurality of tasks;
configuring the number and/or order of tasks;
configuring one or more tasks, optionally as to the timing such as duration or pacing, extent, appearance, accuracy, complexity, trajectory, and/or other characteristics of physical motion or other real-life behavior required from the user in the physical world to advance the execution of the tasks regarding the virtual content (i.e. performance evaluation criteria);
configuring at least one therapeutic session and/or module comprising virtual content and preferably one or more tasks associated therewith;
selecting a virtual representation of a task in a virtual or virtually augmented environment from a plurality of options;
configuring a virtual representation of a task in a virtual or virtually augmented environment (and/or particularly in a virtual space forming a part of the virtual environment);
selecting a virtual environment/space from a plurality of virtual environments/spaces;
configuring a virtual environment/space;
configuring one or more virtual objects illustrated in a virtual environment/space or virtual part of the virtually augmented environment, optionally type, size, color, rotation, (translational) movement, position, and/or location, based on the measurement data; and
configuring user-activating content and/or mutual order, proportion, transition, or other relationship between the user-activating content and behavior-change content.

Configuration may in the above refer to adapting an existing item or entity, or defining a new one, for instance. As being appreciated by a person skilled in the art, the list is not intended exhaustive by any means.

In addition to or instead of dynamically determining the therapeutic program in terms of user-activating content and related elements as discussed above, in various embodiments the dynamic determination may comprise adapting the virtual content and/or related elements of the domain involving behavior-change content by e.g. any of the determination/adaptation options listed above (consumption of behavior-change content and/or user response to it, or user behavior in conducting possible associated tasks involving e.g. exposure therapy or other forms of CBT, is preferably also tracked by the measurement data in addition to or instead of user-activating content related behavior), and/or mutual order, proportion, or other relationship between content types of such domain or between such domain and the domain involving user-activating content in the therapeutic program. Content of any domain (e.g. behavior-change, user-activating) could also be adapted or otherwise dynamically determined based on the user's response or performance, or generally measurement data, having regard to content or specifically tasks relating to other domain.

The arrangement may be configured to alternately and/or simultaneously provide virtual content from at least two domains of virtual content of the therapeutic program. This may be based on the measurement data and/or control input by the user. For example, the measurement data may be used to analyze the user's status and/or task-related (real world) performance to adjust content provision such as content (type/domain) proportions, selection and/or switching.

In more detail and as discussed hereinbefore, the user may deliberately enter a virtual space of certain virtual content in the overall virtual environment according to their mood, for instance.

On the other hand, the arrangement may be configured to adapt the proportion of different content types within and/or between content domains based on e.g. the measurement data. For instance, if the user is expressing certain real-life/physical world status or condition (i.e. non virtual, but still potentially at least partially psychological status) such as excessive physical and/or psychological exertion, dissatisfaction or e.g. excessive fear according to the criteria targeted to the measurement data, proportion of content during the exposure of which such measurements were executed may be reduced at least temporarily in the therapy program in favor of other content type from the same or different domain. For example, fear confrontation type content or user-activating content could be reduced in favor of relaxational content in case the measurement data indicates excessive fear or physical fatigue/exertion, respectively.

In various embodiments, at least partially subjective (measurement) data such as data obtained from the user (e.g. Tampa Kinesiophobia Scale based and/or other (questionnaire) data) may be utilized to assess the progress of the user in the therapeutic program optionally together with e.g. sensor-based more objective measurement data. A patient in pain can be assigned with e.g. relaxational content or other behavior-change content over user-activating content (or correspondingly directed from the activity space of the virtual environment to the safe space, for example).

In various embodiments, the arrangement may be configured to dynamically adapt the therapeutic program such as the virtual content of any domain responsive to time spent by the user in, for example, using the arrangement, accessing the virtual content of the therapeutic program or selected domain(s) thereof, or generally participating in the therapeutic program. For example, when the time spent exceeds an original schedule, the amount of encouraging content (e.g. verbal reinforcement/encouraging messages) may be elevated to motivate goal relevant behavior, which may get the user to better keep up with the schedule. Additionally or alternatively, the virtual content may be adapted so as to indicate easier to complete tasks.

In various embodiments and in the light of the foregoing, an ongoing or planned therapeutic program comprising VR/AR virtual content may be thus adapted in the dynamic determination affecting the associated virtual content in a variety of ways. For example, virtual content from different domains may be selected, timed and/or their amount or mutual portions adapted by the arrangement based on e.g. the status and/or performance of the user as indicated by the measurement data and/or other input. Yet, the adaptation may be based on the nature of the medical condition of the user and/or associated objective of the therapeutic intervention to be provided by the arrangement. The necessary linkage between such information elements may be stored in the arrangement (memory element such as database(s)) and/or external elements such as a remote database that are functionally connected to, such as accessible by, the arrangement. The control system of the arrangement may be configured to execute the adaptation dynamically, optionally even substantially in real-time fashion during the use of the arrangement by the user. For example, if the user fails to perform a task as indicated to them by the user-activating content, the portion of the behavior-change content of especially encouraging type (e.g. encouraging visual and/or audible message) may be provided to the user either together with the user-activating content or instead of it.

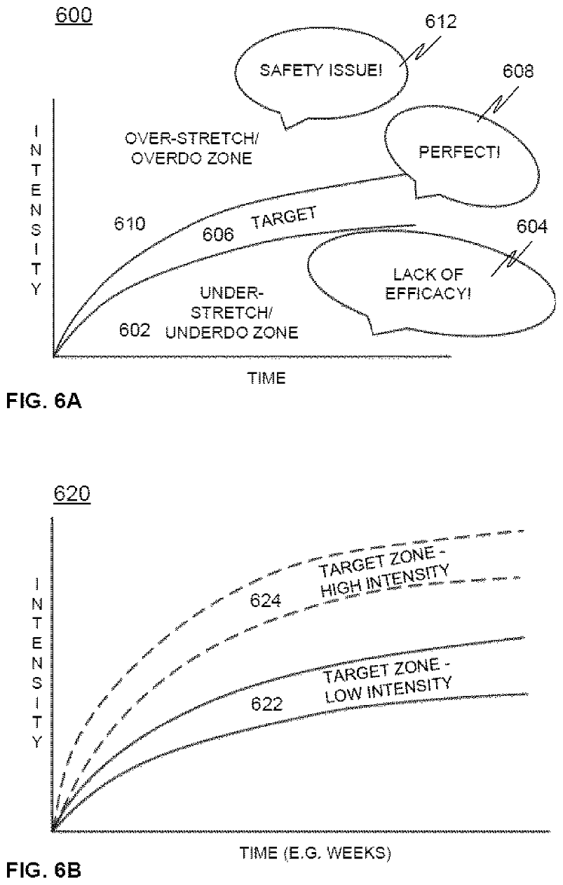

FIG. 6A illustrates, at 600, an embodiment of a therapeutic program including virtual content and e.g. related guidelines for evaluating the user's performance in conducting the tasks through associated therapeutic behavior such as movement in the physical world (real world) relative to time.

Horizontal axis refers to time elapsed (depending on the embodiment, the overall time including only the duration of the VR/AR experience or the actual therapy therewithin, or also e.g. the idle/passive periods in between) and vertical axis to the intensity of the user's physical world behavior in conducting the tasks as measured by the monitoring equipment, for instance. The sweet spot intensity (range) is configured to evolve over time as well as corresponding overdoing and underdoing zones.

At any instant, the behavior of the user may be measured and compared with the zones/thresholds set by the program to estimate the user's performance. In case the user overperforms or overdoes the therapeutic behavior such as over-stretches in the case of range of motion enhancing tasks, the related task may be adapted or new task configured so as to require lesser effort to reduce the risk over overdoing and/or the user may be provided with content of different type such as relaxational and/or instructional content (e.g. in the safe zone type virtual space). In the case of detected sweet spot behavior, the behavior may be reinforced and rewarded by the arrangement by e.g. supportive messages and high score/evaluation report in a gamification style implementation. In the case of underdoing, the user may be provided with encouraging and motivating content e.g. in the safe zone of the virtual environment.

In various embodiments, the user's behavior exceeding or falling short of a selected threshold in conducting a task or the series of tasks may be translated into configuring the task more or less demanding or selecting a more or less demanding task (e.g. in terms of the required therapeutic behavior converted into associated activity in the virtual environment), and/or updating an estimate of the medical condition of the user according to a selected translation logic.

In terms of temporal dimension, as the user is monitored during the VR/AR sessions and preferably also otherwise as discussed hereinelsewhere, also the time spent using the arrangement or spent e.g. with the therapeutic program related activities such as sessions and/or tasks may be monitored. It may happen that based on the available data indicative of the status of the user and optimum timing for progression in the context of the concerned medical condition and e.g. ongoing therapeutic program, changes in the user's behavior or performance take place too fast or slowly instead of optimal pace. Indeed, e.g. (wearable) sensors of the monitoring equipment 114 or results provided in more subjective type of data based on e.g. questionnaire data and/or Tampa Kinesiophobia Scale may be compared against selected criteria to determine and potentially act upon this.

The arrangement may then adjust e.g. the timing aspects of the therapeutic program itself. This may incorporate as discussed hereinbefore, temporally extending or shortening the overall duration of therapy or related constituent elements involving e.g. various content domains, such as conduction of tasks in user-activating domain and/or behavior-change domain to achieve more optimum performance.

Accordingly, compliance with the therapeutic program may be determined. If progress is too slow, it may be that the arrangement is not used as much as prescribed (lack of compliance with the therapeutic program). Treatment failure could thus occur. The user may be additionally encouraged, e.g. with supporting messages, for better compliance in the future. The encouragement may be provided preferably from within the virtual environment such as the safe zone or other space incorporating behavior-change content. Alternatively or additionally, the program may be adapted by including more and/or more advanced (more demanding in terms of the associated required therapeutic behavior) tasks therein e.g. in the domain of user-activating content. If the progress is too fast, the user may also be instructed to follow the program more carefully (if the problem resides in non-compliance) and/or the program may be altered in favor of slower progression (less sessions, shorter sessions, etc.).

Generally, e.g. the safe zone may offer or lead towards a plurality of therapeutic interactions as already discussed hereinbefore with reference to FIGS. 3A and 3B. It may be advantageous to prevent the user from participating in too many or wrong therapeutic sessions at a certain instant or during a certain time interval. Instead, in accordance with the therapeutic program and related objectives certain modules/sessions/content may be repeated over time to ensure learning takes place and other modules/sessions may be introduced. Some modules/sessions will be more helpful than others in certain situations and can be assigned more frequently to optimize the user's experience and development, for instance. The arrangement may be configured to shut down, lock out, hide some modules/sessions/content from the user or prevent accessing them to ensure proper use of the arrangement in favor of the user. However, based on a control signal assigned by e.g. healthcare professional, such shut down or similar state may be cancelled or the user's therapeutic program adapted to enable execution of previously inaccessible content.

FIG. 6B further illustrates, at 620, dynamically determining (adapting or initially defining) the therapeutic program as provided by the electronic arrangement in terms of the intensity of associated therapeutic behavior relative to time. The intensity may refer to e.g., intensity of real life physical tasks in the light of related energy consumption and/or the extent of movement. In the case of mental activity required the extent of mental exertion could be considered, for instance.

The program adjustments may be done e.g. in response to related explicit control input from a healthcare professional or the user, or to other data such as measurement data obtained via various sensors.

Two 622, 624 merely exemplary variations of a therapeutic program are shown with different characteristic task intensities or generally difficulties from the standpoint of associated required physical world (therapeutic) behavior, while the ultimate target objective (treating a certain medical condition) and/or the mechanism of treating it (the nature of the VR/AR tasks and related target physical behavior) might still be substantially the same in both variations of the program. With the higher intensity tasks, the duration of the therapeutic program may be selected or adjusted shorter, for example, and vice versa.

In various embodiments, responsive to control input indicative of a user-preference, a lower or higher intensity therapeutic program may be selected with characteristic target zones for the intensity (see also the previous discussion regarding FIG. 6A).

Advantageously, the arrangement is configured to monitor the user based on e.g. sensor-based objective and/or subjective measurement data and dynamically (re)determine intensity-optimized therapeutic program to the user or at least suggest such via communication with the user e.g. in the safe zone or other space of the VR/AR experience or outside it.

In various embodiments, the arrangement may be configured to increase e.g. the duration of the therapeutic program and lower the estimated difficulty of the included series of tasks from the standpoint of associated therapeutic behavior required to advance the tasks, or vice versa. Adaptation of the difficulty of one or more tasks may naturally involve in this and other embodiments adapting at least the evaluation criteria for the virtual tasks or in particular, therapeutic behavior required to take place in the physical world to advance the task(s), adapting the timing of tasks (e.g. time limits) for successfully executing them via the associated behavior in the physical world, and/or adapting the nature of tasks and/or of the associated behavior more thoroughly, for example.

With reference to different medical conditions and the applicability of various embodiments of the present invention for treating those, two further examples are constructed below for potential additional case-specific adaptation by a person skilled in the art (the person skilled in the art shall also acknowledge the fact that the principles set forth below may be selectively utilized in treating other medical conditions as well):

Kinesiophobia caused by low back pain:

Kinesiophobia is here broadly defined as fear of increased pain, of increased harm, or of re-injury due to movement, which may lead to a pattern of avoidance of specific and general behavior, which can in turn hinder rehabilitation and prolong disability and pain.

One feasible type of subjective input data here to the arrangement is user scoring on the known Tampa Kinesiophobia Scale or other applicable scale. The degree of kinesiophobia is calculated as a non-dimensional number, where a medium score is represented by score of 34-41 and a high score on the TKS is 42-68. The arrangement may be thus configured to assess the degree of kinesiophobia and adapt the therapeutic program accordingly. In addition, individual questions may be factorized and provide data on sub-components. If, for example, the user scores high on questions such as “My body is telling me I have something dangerously wrong” and low on “Just because something aggravates my pain does not mean it is dangerous”, the arrangement may be configured to direct content to the user, e.g. in the safe zone or other virtual space of behavior-change content, providing additional reassurance and cognitive reasoning. If the user scores high on questions such as “Pain lets me know when to stop exercising so that I don't injure myself” and low on “Even though something is causing me a lot of pain, I don't think it's actually dangerous”, the arrangement may be configured to address extra movement excursions (range of motion task) by suitable user-activating content to the user to stretch the user and demonstrate no harm. This may be achieved, as discussed hereinbefore, by determining the variance of the physical extremes of movement of e.g. hand-controllers carried and headset worn by the user; an extra excursion beyond normal limits will then be programmed (e.g. at random) to the user to reach. For example, game theory will provide the motivation.

Another form of input data may be movement data indicative of e.g. the number of steps and distance walked, which may be measured by the sensors of the monitoring equipment, for instance. These data may be used to estimate the physical movement and e.g. concordance of physical movement with the planned goal-determined activity (tasks assigned and to be performed), and/or the influence of fear-avoidance and fear of movement beliefs in the actual performance achieved. The data may be correlated to the psychological score obtained by e.g. the Tampa Scale, for instance. Further, the user's self-awareness and self-reporting may be estimated. A healthcare professional consulting the user may give this feedback to the user optionally via the communication features of the arrangement, or such information may be delivered based on fully automated determination and delivery.

Over time assessment of specific fear-relevant barriers to goal relevant behavior may be recognized and analyzed and the therapeutic program adapted in order to keep delivering on the required components. For example, the obtained scores may alter and the associated trends be identified. One feasible objective of the therapeutic program is in this scenario a Tampa score below 34.

Restricted arm movement caused by Complex Regional Pain Syndrome:

Complex Regional Pain Syndrome (CRPS) Type 1 is a physician diagnosis. The most promising current theory is that crush injury to a nerve exposes antigens not normally available to or surveyed by the patient's immune system (neoantigens). An autoimmunological reaction occurs which has an IgG+/−IgM component in serum; this causes the signs and symptoms of CRPS. Patients complain of severe pain mostly unrelieved by current therapies, changes in skin temperature, skin color, and/or swelling of the affected limb.

The arrangement preferably calculates the movement of e.g. two hand controllers (one in each hand again) in relation to the headset worn by the user. This gives accurate estimation of the (“envelope” of) hand movement, with reference also to the vector and volume curve determinations discussed hereinbefore. CRPS usually affects one limb only; thus the control reference can in this case be the good arm. The arrangement may be configured to inquire from the user which arm is normally dominant, whereupon it (taking the answer into account) it sets the tasks via user activating type content to encourage movement of the afflicted arm. The user may be encouraged by real human or e.g. AI/software-based mentor in the safe zone or other virtual space of behavior-change content. The used vocabulary may be altered to focus on CRPS patients and hand movements, which applies also to other embodiments of the present invention (communicational and/or other content provided to the user may be adapted based on the medical condition and/or the nature of the therapeutic program of the user, utilizing e.g. translation table, other data structure(s) and/or programmatic translation logic for the purpose).

A feasible objective of the therapeutic program is in this scenario to achieve movement in the afflicted arm equal to that in the good arm, for instance.

Having regard to the safety of the user in consuming the VR/AR content in various embodiments of the present invention, the arrangement is preferably configured to, based on an indication of optionally physical and/or mental capacity of the user as preferably indicated by the measurement data (objective and/or subjective as discussed hereinbefore), to
determine the therapeutic program so that the therapeutic behavior required to advance the tasks remains within the capacity or exceeds it by a selected amount only; and/or to
notify the user when the capacity limit is approached, reached, or exceeded.

For instance, harmful movements such as overstretching can be controlled (reduced or avoided) by setting the height and reach of the (virtual) target objects properly in the user activating content, to better match the current capabilities of the user.

Based on e.g. previously discussed baseline data or status/characteristic data regarding the user (indication, height/weight/age, and treatment plan, for example) and/or movement range assessment or calibration, a number of safety ranges may be calculated and used in determining the therapeutic program and e.g. related tasks or task evaluation criteria. These factors will help guiding users from not doing too heavy/hard exercises and/or too fast movements in the physical world, for example.

In various embodiments, a limited floor area, within which the user must stay, may be defined before starting the VR/AR experience. This is required in order to avoid hazards such as open fires, stairs, glass tables, wall, windows etc. Several mutually compatible methods can be used for the purpose.

In one method, a piece of non-slip, textured carpet or other element is used to define the safe floor area for movement. The user can easily tell if they have stepped off the element.

In another method, the VR/AR reproduction equipment such as headset incorporates a (virtual) boundary feature; if the user exceeds the limits of the boundary based on e.g. sensor data, the headset switches from virtual reality to actually seeing the real surroundings and/or notifies the user visually and/or audibly. The boundary may be defined, among other options, based on physical obstacles detected in the (physical) space where the VR/AR equipment is used or on predefined operation area limits (e.g. movement or distance related limits from a central/origo position).

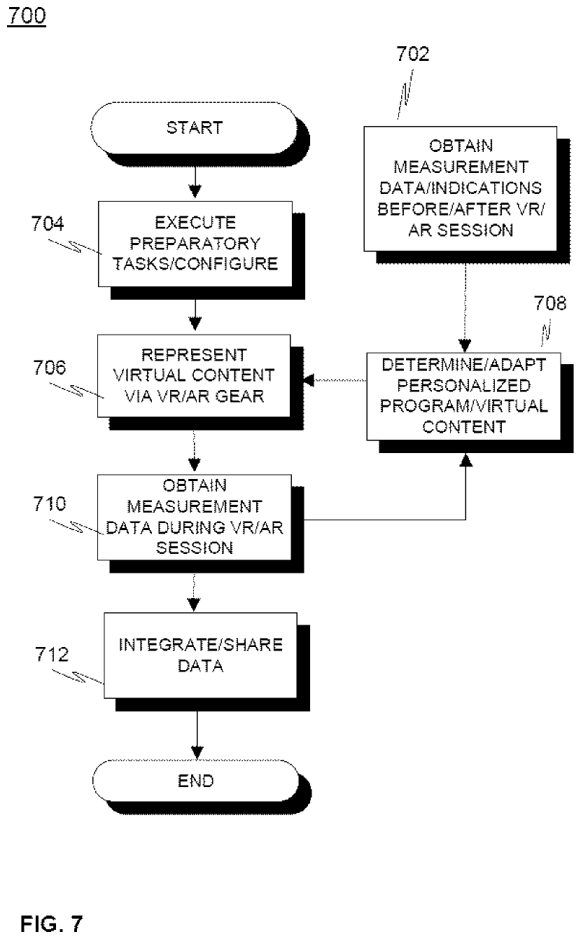

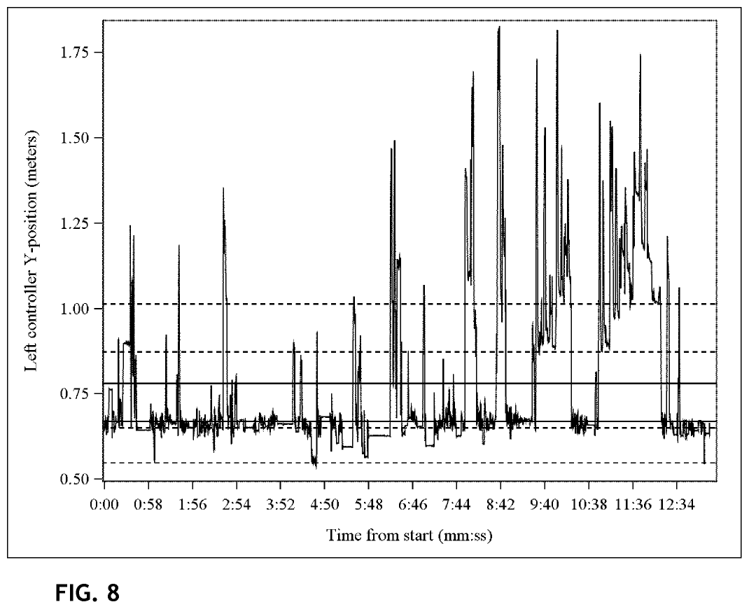

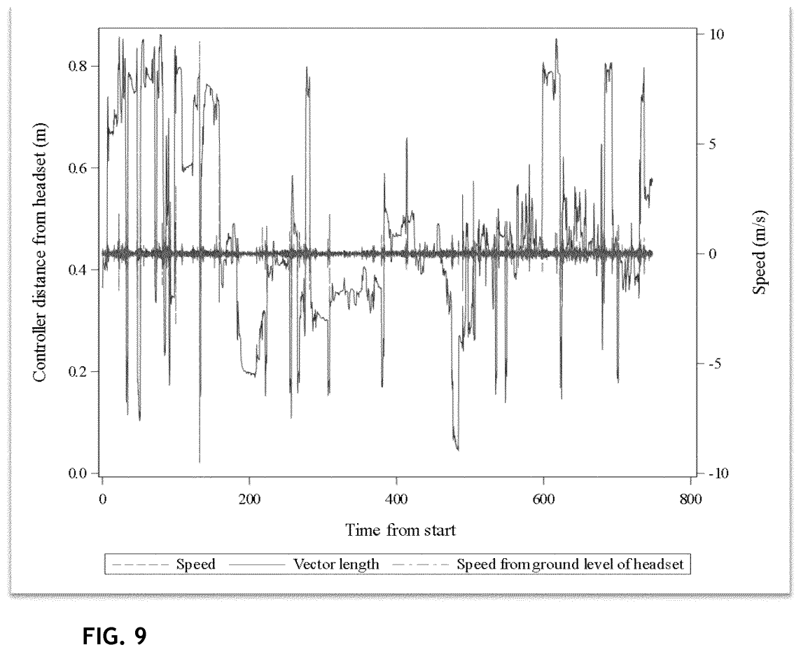

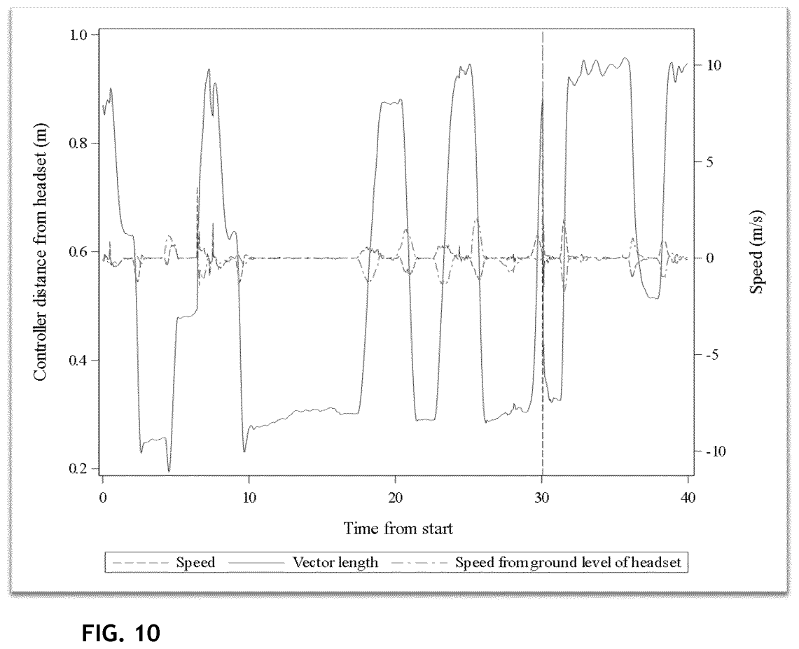

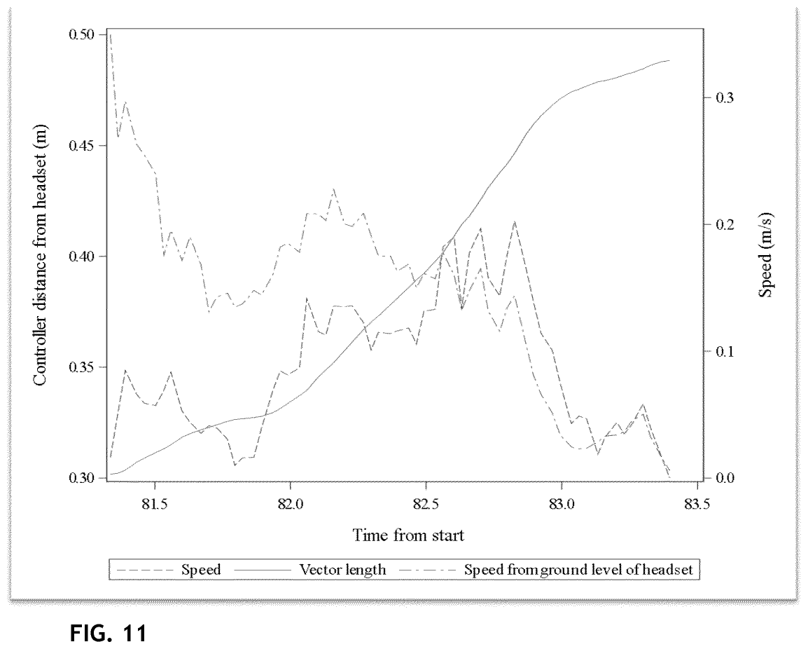

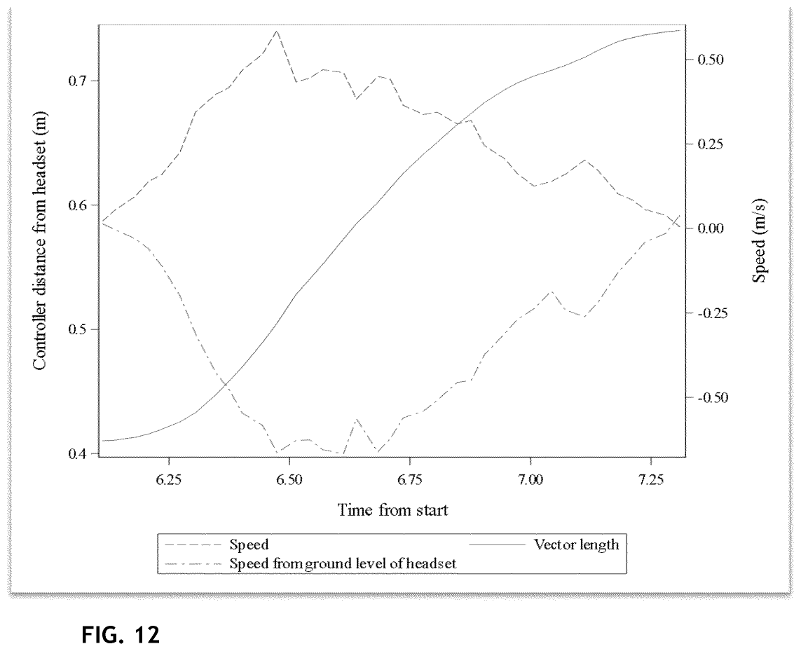

FIG. 7 is a flow diagram 700 disclosing an embodiment of a method in accordance with the present invention for providing therapeutic intervention to a user suffering from a medical condition through the application of virtual reality (VR) or augmented reality (AR). The method is preferably carried out by an embodiment of an electronic arrangement as described hereinearlier.

Although the shown diagram contains a plurality of definite method items or steps, in various other embodiments all the same items do not have to present. There may be additional method items as well not shown in the figure. Depending on the embodiment, some existing method items may be also realized as combined. Yet, the ordering of items may vary between the items and/or their execution may overlap. The execution of shown items such as items 702, 706, 708, 710 and 712 may also be repeated, which is indicated in the figure by a dotted loop-back arrow.

At 704, different preparatory and initial tasks may be executed. For example, an electronic arrangement for executing the method and associated remote entities such as connected devices or systems may be acquired, calibrated and otherwise configured by installing thereat e.g. the necessary hardware elements and/or software. The software may be stored e.g. as a computer program in a memory of a target device and when executed by at least one processing unit causing the device to perform the programmed method items as contemplated hereinbefore. A user account may be created for the user to log into and use within the arrangement and optionally in connected systems or devices, with necessary information (e.g. credentials). A therapeutic program be initially determined for the user based on e.g. automated measurements, healthcare professional(s) conducted measurements and/or subjective or other data obtained concerning the user as deliberated hereinbefore. Desired communication connections may be established and tested.

At 706, the arrangement provides virtual content comprising immersive virtual environment or a virtual part of a virtually augmented environment to the user via reproduction equipment comprising a VR and/or AR projection device as discussed hereinbefore.

Item 710 refers to obtaining measurement data essentially during the VR/AR experience while item 702 refers to obtaining similar and/or different measurement data during other times, optionally utilizing at least partially different equipment for the purpose. Also these topics have been thoroughly discussed hereinbefore.

During or after a therapy session, a prevailing status/condition of the user and e.g. related advancements relative to the original objective of the therapeutic program such as range of movement may be measured either by requesting the user to redo the initial calibration activities, or by including tasks that will incorporate this into the therapeutic program at item 708, for instance.

At 708, the personalized therapeutic program including the virtual content for representation via the reproduction equipment, is dynamically determined (selected or configured through definition or adaptation, for example) based on the measurement data. The executing arrangement may be configured to dynamically determine the therapeutic program such as associated content or tasks so as to facilitate the user to reach the objective of the therapy both safely and motivationally, if not also rapidly. Preferably, the content provided by the arrangement during the therapeutic program comprises both behavior-change and user-activating content, but in some use scenarios only one content type/domain could be relied upon.

As being already extensively reviewed hereinbefore, dynamic determination of the therapeutic program may temporally happen prior to starting the execution of the program and/or during the execution, which may refer to the periods of actual VR/AR experience or actual therapy, and optionally the idle/passive periods in between the sessions of VR/AR experience or actual therapy.

Item 712 refers to various possible other tasks such as data integration (e.g. derivation of e.g. aggregate statistics regarding one or multiple users for monitoring, storage, or analysis such as status/performance assessment optionally for creating control or support data for use in the dynamic determination of the therapeutic program), sharing (to external systems/devices of e.g. healthcare professionals or other stakeholders) and input (e.g. control input from external systems/devices of therapeutic professionals or other entities) activities that may be performed among others during the execution of the method. Item 712 may be intermittently or substantially continuously executed simultaneously with any of the remaining method items.

In various embodiments of the present invention the status or condition of the user may be monitored, besides prior to or during participation in a therapeutic program as discussed hereinbefore, also afterwards either for a selected or indefinite period. The user monitoring equipment (e.g. items 114B) may be optionally used for this purpose as well. Yet, subjective/self-reported data such as questionnaires, diaries or free format input may be gathered in addition to or instead of more automated acquisition of objective, typically sensor-based, data.

In cases where the user keeps on using the arrangement more thoroughly after finishing the associated therapeutic program (with reference to both items 114A and 116, i.e. VR/AR reproduction and related monitoring gear as well in addition to e.g. more generic sensor/monitoring items 114B), the user may, through the execution of related recalibration activities or other tasks also preferably performed prior to or during the therapy, provide measurement data even for comprehensive user status/condition evaluation also afterwards.

Based on the obtained data, status or condition of the user after the therapy may be compared with the situation prior to or during the treatment, and thus indicator(s) of the treatment's immediate and/or long-lasting effects be conveniently obtained. The achieved movement related results such as the range of motion may be verified against pre-therapy situation as well as the results regarding e.g. phobias or sensation of pain, with reference to previous discussion of how to measure such issues using e.g. biometric quantities such as vital signs related data.

If the data collected shows e.g. potential deterioration of the user's condition or the symptoms getting worse, the arrangement may be configured to automatically trigger, based on the selected criterion regarding e.g. subjective data or sensor data or indicators determined based on the (other) available data, a number of responsive action(s) such as notifying the user and/or healthcare professional about the situation. The user may be instructed to start a new therapeutic program by the arrangement, for example.

Also disclosed are an electronic arrangement, method and computer program product according to any of the following items:
1. An electronic arrangement (100) for use (200) in providing therapeutic intervention to a user (201) suffering from a medical condition, optionally to reduce fear of movement and improve function in a user with chronic pain, via virtual reality (VR) or augmented reality (AR), comprising
   a reproduction equipment (116) comprising a VR and/or AR projection device configured to represent virtual content, comprising an immersive virtual environment or a virtual part of a virtually augmented environment, to the user;
   user monitoring equipment (114, 114A, 114B) configured to obtain measurement data regarding the user, including motion, location, position, and/or biometric data; and
   a control system (118, 118A, 118B, 118C), at least functionally connected to the reproduction equipment and the user monitoring equipment, and configured to dynamically determine a personalized therapeutic program including the virtual content for representation via the reproduction equipment, based on the measurement data,
   wherein the therapeutic program comprises at least two domains of different virtual content, one or more of the domains involving behavior-change content (300, 310, 410, 500) and at least one other domain involving user-activating virtual content (400, 500) indicative of a series of tasks (404, 406, 408) to be conducted by the user having regard to the virtual content through associated therapeutic behavior, such as physical activity, in the physical world outside the virtual environment or virtually augmented environment and tracked by the measurement data.
2. The arrangement of any preceding item, configured to track the user's behavior, optionally biometric response, relative to the behavior-change content based on the measurement data, wherein the behavior-change content is optionally associated with said or other series of tasks and/or target therapeutic behavior for the user to reach responsive to perceiving the content, and preferably to utilize it in said dynamic determination of the therapeutic program, such as in the adaptation of the behavior-change content.
3. The arrangement of any preceding item, configured to estimate the performance of the user in conducting the series of tasks by subjecting the measurement data indicative of the behavior of the user to a number of performance evaluation criteria indicative of the therapeutic behavior required to advance the series of the tasks, and based on a resulting estimate of the performance, execute the dynamic determination of the personalized therapeutic program, optionally comprising adapting the virtual content of any of the domains preferably including at least the user-activating domain.
4. The arrangement of any preceding item, configured, based on an indication of the capacity of the user preferably indicated by the measurement data, to
   dynamically determine the personalized therapeutic program so that the therapeutic behavior required to advance the tasks remains within the capacity or exceeds it by a selected amount only; and/or to
   notify the user when the capacity limit is approached, reached, or exceeded.
5. The arrangement of any preceding item, configured to compare first measurement data or data derived therefrom relating to a first body part with second measurement data or data derived therefrom relating to a second body part and/or a reference point, and based on the comparison result to determine an indication of the status, medical condition and/or selected anthropometric, musculoskeletal, physiological or other characteristics of the user, optionally comprising an indication of flexibility or range of motion, and preferably to further dynamically determine the personalized therapeutic program optionally comprising selecting or configuring such as adapting one or more tasks of the series of tasks and/or the extent of associated therapeutic behavior needed to advance the tasks,
   wherein the first measurement data or data derived therefrom preferably concerns head, trunk or first limb of the user and the second measurement data or data derived therefrom preferably concerns at least second limb of the user, said first and second measurement data further preferably comprising movement data.
6. The arrangement of any preceding item, configured to represent, visibly and/or audibly, a computer-generated, preferably artificial intelligence based, virtual therapist with a characteristic visual appearance, optionally a graphic figure such as an avatar, and/or voice to the user via the reproduction equipment, and configured to provide the user with instructions, support or feedback, optionally regarding the use of the arrangement or the therapeutic program, via the virtual therapist.
7. The arrangement of any preceding item, configured to increase the duration of the therapeutic program and lower the estimated difficulty of the included series of tasks from the standpoint of associated therapeutic behavior required to advance the tasks, or vice versa, responsive to control input preferably indicative of a related user-preference.
8. The arrangement of any preceding item, configured to
   provide a real-time and/or non-real-time communication channel or platform between the user and at least one other human party, optionally a health care professional and/or other user of the same or a functionally connected other arrangement, preferably in the virtual environment wherein one or more of the communicating parties are represented graphically optionally by avatars; and/or
   store and indicate, preferably via the reproduction equipment, the user's performance in meeting an objective preferably comprising conducting the series of tasks, preferably against the user's previous performance and/or the performance of a number of other users.
9. The arrangement of any preceding item, wherein the dynamic determination comprises at least one element selected from the group consisting of:
   selecting a task from a plurality of tasks;
   configuring the number and/or order of tasks;
   configuring one or more tasks, optionally as to the timing such as duration or pacing, extent, appearance, accuracy, complexity, trajectory, and/or other characteristics of physical motion or other real-life behavior required from the user in the physical world to advance the execution of the tasks regarding the virtual content;
   configuring at least one therapeutic session and/or module comprising virtual content and preferably one or more tasks associated therewith;
   selecting a virtual representation of a task in a virtual or virtually augmented environment from a plurality of options;
   configuring a virtual representation of a task in a virtual or virtually augmented environment;
   selecting a virtual environment from a plurality of virtual environments or a virtual space within the virtual environment from a plurality of spaces;
   configuring a virtual environment or virtual space within the virtual environment;
   configuring one or more virtual objects (302, 304, 306, 312, 314, 316, 318, 404) illustrated in a virtual environment or virtual part of the virtually augmented environment, optionally their type, size, color, rotation, translational movement, position, and/or location, based on the measurement data;
   configuring behavior-change content and/or mutual order, proportion, transition or other relationship between multiple types of behavior-change content or between behavior-change content and user-activating content; and
   configuring user-activating content and/or mutual order, proportion, transition or other relationship between the user-activating content and behavior-change content.
10. The arrangement of any preceding item, wherein the virtual content indicative of the series of the tasks to be conducted visualizes one or more virtual target objects (404) reaching, manipulation or other addressing of which in the virtual environment or virtual part of a virtually augmented environment enables to achieve the tasks,
   preferably wherein at least one virtual target object (404) defines a preferably geometric shape, optionally a tetromino, polyomino, tetracube, polycube or alike, to be preferably manipulated by rotation, translational movement, introduction, removal, resizing or reshaping in the virtual environment or virtual part of the virtually augmented environment, optionally through conducting similar or other activity associated therewith in the physical world by the user and indicated by the measurement data.
11. The arrangement of any preceding item, configured to dynamically determine the personalized therapeutic program and/or specifically adapt the virtual content of any domain responsive to time spent by the user in using the arrangement, accessing the virtual content of the therapeutic program or selected domain(s) thereof, or generally participating in the therapeutic program.
12. The arrangement of any preceding item, wherein the user monitoring equipment comprises at least one element selected from the group consisting of: personal computer, mobile terminal, wearable electronic device, wristop device, control input sensor, accelerometer, gyroscope, inertial sensor, camera, optical sensor, location sensor, position sensor, temperature sensor, moisture sensor, pressure sensor, distance sensor, eye sensor, implantable sensor, biometric sensor, motion sensor, and a microphone.
13. The arrangement of any preceding item, wherein the control system (118) comprises a first sub-system (118A), optionally being at least partially integral with the reproduction equipment and/or user monitoring equipment, and at least a second sub-system (118B, 118C) remote from but functionally connected, optionally via the internet, to the first sub-system, wherein
   the first sub-system is configured to process measurement data retrieved from the user monitoring equipment and provide at least portion of the processed data to the second sub-system for further processing, storage and/or determination of at least portion of the therapeutic program or related attributes thereat;
   the second sub-system is configured to obtain measurement data and/or data derived therefrom from the user monitoring equipment and/or the first sub-system, and to process, store and/or determine at least portion of the therapeutic program or related attributes based on the obtained data; and/or
   the first sub-system is configured to receive information such as attributes determining the therapeutic program from the second sub-system and to utilize it for controlling the VR reproduction equipment to represent virtual content in accordance with the therapeutic program:
   preferably wherein the first sub-system is configured to autonomously determine the therapeutic program and/or control the reproduction equipment to represent virtual content in accordance with the therapeutic program based on the measurement data, responsive to fulfillment of at least one selected condition such as connection failure between the first and second sub-systems.
14. The arrangement of any preceding item, configured to obtain, optionally via the user monitoring equipment (114, 114A) and/or healthcare professional operated control interface (124),
   user-created subjective data, such as questionnaire, note or diary data, characterizing the status, characteristic and/or condition, such as mental or physical condition, of the user and to utilize the user-created subjective data in dynamically determining the therapeutic program; and/or
   healthcare professional-provided subjective data characterizing the status, condition, behavior and/or task related performance of the user, and to utilize the healthcare professional-provided subjective data in dynamically determining the therapeutic program and/or comparing and optionally mutually verifying the healthcare professional-provided data in relation to other data preferably including automatically created sensor-based measurement data or user-created measurement data.
15. The arrangement of any preceding item, configured to utilize a selected machine learning algorithm in determining the therapeutic program, said machine learning algorithm associating sensor-based objective measurement data and/or subjective data, or data derived therefrom, with the therapeutic program or interim result to be utilized in determining the therapeutic program.
16. The arrangement of any preceding item, configured to obtain, utilizing said user monitoring equipment, measurement data regarding the user during periods outside the consumption of the virtual content, said obtained measurement data preferably comprising at least one data element selected from the group consisting of: user activity information, call data, messaging data, communication data, physical activity or passivity data, sleep data, insomnia data, social media activity data, motion, motoric, location, position, and/or biometric data.
17. The arrangement of any preceding item, wherein the virtual content indicative of the series of tasks to be conducted visualizes the nature, progress, goal, outcome and/or execution of the associated therapeutic behavior, such as physical activity, to be performed by the user to advance the execution of one or more of the tasks, optionally utilizing one or more alphanumeric characters, symbols, pictures, or animations.
18. The arrangement of any preceding item, configured to alternately or simultaneously provide virtual content from at least two domains of virtual content of the therapeutic program, optionally based on the measurement data and/or control input by the user.
19. The arrangement of any preceding item, wherein the virtual content indicative of the series of the tasks to be conducted comprises audio data.
20. The arrangement of any preceding item, configured to alter the user's, or of a corresponding virtual character's or pointer's, position, location, rotation or translational speed, and/or viewing direction in a virtual environment or virtually augmented environment based on the measurement data optionally indicative of the user's volitional control input captured through one or more sensors of the user monitoring equipment.
21. The arrangement of any preceding item, comprising a haptic device configured to provide haptic sensations to the user preferably at least responsive to contacting a virtual object in the virtual or augmented environment.
22. A method (700) for providing therapeutic intervention to a user suffering from a medical condition by an electronic arrangement through the application of virtual reality (VR) or augmented reality (AR), comprising:
   providing virtual content (706) comprising immersive virtual environment or a virtual part of a virtually augmented environment to the user via reproduction equipment comprising a VR and/or AR projection device;
   obtaining (702, 710) measurement data, via user monitoring equipment, regarding the user, including motion, location, position, and/or biometric data; and
   dynamically determining (708) a personalized therapeutic program including the virtual content for representation via the reproduction equipment, based on the measurement data,
   wherein the therapeutic program comprises at least two domains of different virtual content, one or more of the domains involving behavior-change content and at least one other domain involving user-activating virtual content indicative of a series of tasks to be conducted by the user having regard to the virtual content through associated therapeutic behavior, such as physical or problem solving activity, in the physical world outside the virtual environment or virtually augmented environment and tracked by the measurement data.
23. A computer program product optionally embodied in a preferably non-transitory computer-readable carrier medium, said program comprising instructions, which, when the program is executed by a computer, cause the computer to carry out an embodiment of a method of item 22.

The general scope of various aspects of the present invention is defined by the attached independent claims with appropriate national extensions thereof having regard to the applicability of the doctrine of equivalents. Although the embodiments explicitly described in this document mainly concerned especially virtual reality (VR) type solutions, a person skilled in the art will readily implement the solution mutatis mutandis also in the context of augmented reality (AR) based on the provided information.

### Example 1

Feasibility study (VR) cohort 1 patients and healthy volunteers

Data sources:

8 subjects was enrolled, but one healthy subject subsequently excluded due to low number of identifiable movements (4 in total). Thus the study included 7 subjects data used: n=2 chronic lower back pain, n=3 chronic pain, n=2 healthy Duration of range of motion (ROM) data measured from the accelerometers (left and right controller and headset) ranged from 748 seconds to 2193 seconds (only 78 seconds for the one excluded subject).

From these subjects (n=7) ROM data, a total of 1579 pushing movements (distance between headset and hand controller increasing) and 1569 drawing movements (distance between headset and hand controller decreasing) was detected. Of each individual movement we have calculated mean speed, std of mean speed, and number of speed changes per second during a movement was calculated.

Test data for standard movement routine (approximately 45 seconds) was prepared using a group of healthy volunteers n=17.

This testing was conducted originally to explore the different options of frequencies for ROM data collection. A standard routine of hand movements was guided to the healthy volunteers who repeated this movement a total of 3 times. From 14 subjects movements could be detected. Number of detected movements ranged from 14 to 51. A total of 485 pushing movements and 498 drawing movements were detected. In addition of these healthy volunteers (n=14), there was 1 additional volunteer with one-sided back pain. 34 pushing movements and 31 drawing movements were detected from this subject.

As a description of move, a limit of 5 cm distance between 2 changes of direction, was applied. Within the move it was allowed to have smaller intermittent changes of direction.

1. An electronic arrangement for use in pain management or for treating or ameliorating kinesiophobia via virtual reality (VR) or augmented reality (AR), comprising:
a reproduction equipment comprising a VR and/or AR projection device configured to represent virtual content, comprising an immersive virtual environment or a virtual part of a virtually augmented environment, to the user;
user monitoring equipment configured to obtain measurement data regarding the user, including motion, location, position, and/or biometric data; and
a control system, at least functionally connected to the reproduction equipment and the user monitoring equipment, and configured to dynamically determine a personalized therapeutic program including the virtual content for representation via the reproduction equipment, based on the measurement data,
wherein the therapeutic program comprises at least two domains of different virtual content, one or more of the domains involving behavior-change content and at least one other domain involving user-activating virtual content indicative of a series of tasks to be conducted by the user having regard to the virtual content through associated therapeutic behavior in the physical world outside the virtual environment or virtually augmented environment and tracked by the measurement data;
wherein the therapeutic program is configured to, based on an indication of the capacity of the user indicated by the measurement data, dynamically determine the personalized therapeutic program so that the therapeutic behavior required to advance the tasks remains within the capacity of the user or exceeds the capacity of the user by a selected amount only;
wherein the therapeutic program is configured to compare first measurement data or data derived therefrom relating to a first body part with second measurement data or data derived therefrom relating to a second body part and/or a reference point, and based on the comparison result to determine an indication of the status, medical condition and/or selected anthropometric, musculoskeletal, physiological or other characteristics of the user;
wherein the first measurement data or data derived therefrom optionally concerns head, trunk or first limb of the user and the second measurement data or data derived therefrom optionally concerns at least second limb of the user; and
wherein said first and second measurement data comprises movement data.

2-61. (canceled)

62. The arrangement of claim 1, comprising an indication of flexibility and/or range of motion to further dynamically determine the personalized therapeutic program.

63. The arrangement of claim 1, wherein said arrangement is configured to track the user's behavior, optionally biometric response, relative to the behavior-change content based on the measurement data, wherein the behavior-change content is optionally associated with said or other series of tasks and/or target therapeutic behavior for the user to reach responsive to perceiving the content, and optionally to utilize it in said dynamic determination of the therapeutic program.

64. The arrangement of claim 1, wherein said arrangement is configured to:
provide a real-time and/or non-real-time communication channel or platform between the user and at least one other human party, optionally a health care professional and/or other user of the same or a functionally connected other arrangement, optionally in the virtual environment, wherein one or more of the communicating parties are represented graphically optionally by avatars; and/or
store and indicate, optionally via the reproduction equipment, the user's performance in meeting an objective optionally comprising conducting the series of tasks, optionally against the user's previous performance and/or the performance of a number of other users.

65. The arrangement of claim 1, wherein the dynamic determination comprises at least one element selected from the group consisting of:
selecting a task from a plurality of tasks;
configuring the number and/or order of tasks;
configuring one or more tasks, optionally as to the timing, duration,
pacing, extent, appearance, accuracy, complexity, trajectory, and/or other characteristics of physical motion or other real-life behavior required from the user in the physical world to advance the execution of the tasks regarding the virtual content;
configuring at least one therapeutic session and/or module comprising virtual content and optionally one or more tasks associated therewith;
selecting a virtual representation of a task in a virtual or virtually augmented environment from a plurality of options;
configuring a virtual representation of a task in a virtual or virtually augmented environment;
selecting a virtual environment from a plurality of virtual environments or a virtual space within the virtual environment from a plurality of spaces;
configuring a virtual environment or virtual space within the virtual environment;
configuring one or more virtual objects illustrated in a virtual environment or virtual part of the virtually augmented environment, optionally their type, size, color, rotation, translational movement, position, and/or location, based on the measurement data;
configuring behavior-change content and/or mutual order, proportion, transition or other relationship between multiple types of behavior-change content or between behavior-change content and user-activating content; and
configuring user-activating content and/or mutual order, proportion, transition or other relationship between the user-activating content and behavior-change content.

66. The arrangement of claim 1, wherein the virtual content indicative of the series of the tasks to be conducted visualizes one or more virtual target objects reaching, manipulation or other addressing of which in the virtual environment or virtual part of a virtually augmented environment enables to achieve the tasks,
optionally wherein at least one virtual target object defines an optionally geometric shape, optionally a tetromino, polyomino, tetracube, polycube or alike, to be optionally manipulated by rotation, translational movement, introduction, removal, resizing or reshaping in the virtual environment or virtual part of the virtually augmented environment, optionally through conducting similar or other activity associated therewith in the physical world by the user and indicated by the measurement data.

67. The arrangement of claim 1, configured to dynamically determine the personalized therapeutic program and/or specifically adapt the virtual content of any domain responsive to time spent by the user in using the arrangement, accessing the virtual content of the therapeutic program or selected domain(s) thereof, or generally participating in the therapeutic program.

68. The arrangement of claim 1, wherein the user monitoring equipment comprises at least one element selected from the group consisting of: personal computer, mobile terminal, wearable electronic device, wristop device, control input sensor, accelerometer, gyroscope, inertial sensor, camera, optical sensor, location sensor, position sensor, temperature sensor, moisture sensor, pressure sensor, distance sensor, eye sensor, implantable sensor, biometric sensor, motion sensor, and a microphone.

69. The arrangement of claim 1, configured to obtain, utilizing said user monitoring equipment, measurement data regarding the user during periods outside the consumption of the virtual content, said obtained measurement data optionally comprising at least one data element selected from the group consisting of: user activity information, call data, messaging data, communication data, physical activity or passivity data, sleep data, insomnia data, social media activity data, motion, motoric, location, position, and biometric data.

70. A method comprising:
at an electronic arrangement including:
a reproduction equipment comprising a VR and/or AR projection device configured to represent virtual content, comprising an immersive virtual environment or a virtual part of a virtually augmented environment, to the user;
user monitoring equipment configured to obtain measurement data regarding the user, including motion, location, position, and/or biometric data; and
a control system, at least functionally connected to the reproduction equipment and the user monitoring equipment, and configured to dynamically determine a personalized therapeutic program including the virtual content for representation via the reproduction equipment, based on the measurement data,
presenting two domains of different virtual content, one or more of the domains involving behavior-change content and at least one other domain involving user-activating virtual content indicative of a series of tasks to be conducted by the user having regard to the virtual content through associated therapeutic behavior in the physical world outside the virtual environment or virtually augmented environment and tracked by the measurement data;
based on an indication of the capacity of the user indicated by the measurement data, dynamically determining the personalized therapeutic program so that the therapeutic behavior required to advance the tasks remains within the capacity of the user or exceeds the capacity of the user by a selected amount only;
comparing first measurement data or data derived therefrom relating to a first body part with second measurement data or data derived therefrom relating to a second body part and/or a reference point, and based on the comparison result determining an indication of the status, medical condition and/or selected anthropometric, musculoskeletal, physiological or other characteristics of the user;
wherein the first measurement data or data derived therefrom optionally concerns head, trunk or first limb of the user and the second measurement data or data derived therefrom optionally concerns at least second limb of the user; and
wherein said first and second measurement data comprises movement data.

71. The method of claim 70, comprising determining an indication of flexibility and/or range of motion to further dynamically determine the personalized therapeutic program.

72. The method of claim 70, comprising:
tracking the user's behavior, optionally biometric response, relative to the behavior-change content based on the measurement data,
wherein the behavior-change content is optionally associated with said or other series of tasks and/or target therapeutic behavior for the user to reach responsive to perceiving the content, and optionally to utilize it in said dynamic determination of the therapeutic program.

73. The method of claim 70, comprising:
providing a real-time and/or non-real-time communication channel or platform between the user and at least one other human party, optionally a health care professional and/or other user of the same or a functionally connected other arrangement, optionally in the virtual environment wherein one or more of the communicating parties are represented graphically optionally by avatars; and/or
storing and indicating, optionally via the reproduction equipment, the user's performance in meeting an objective optionally comprising conducting the series of tasks, optionally against the user's previous performance and/or the performance of a number of other users.

74. The method of claim 70, wherein the dynamic determination comprises at least one element selected from the group consisting of:
selecting a task from a plurality of tasks;
configuring the number and/or order of tasks;
configuring one or more tasks, optionally as to the timing, duration, pacing, extent, appearance, accuracy, complexity, trajectory, and/or other characteristics of physical motion or other real-life behavior required from the user in the physical world to advance the execution of the tasks regarding the virtual content;
configuring at least one therapeutic session and/or module comprising virtual content and optionally one or more tasks associated therewith;
selecting a virtual representation of a task in a virtual or virtually augmented environment from a plurality of options;
configuring a virtual representation of a task in a virtual or virtually augmented environment;
selecting a virtual environment from a plurality of virtual environments or a virtual space within the virtual environment from a plurality of spaces;
configuring a virtual environment or virtual space within the virtual environment;
configuring one or more virtual objects illustrated in a virtual environment or virtual part of the virtually augmented environment, optionally their type, size, color, rotation, translational movement, position, and/or location, based on the measurement data;
configuring behavior-change content and/or mutual order, proportion, transition or other relationship between multiple types of behavior-change content or between behavior-change content and user-activating content; and
configuring user-activating content and/or mutual order, proportion, transition or other relationship between the user-activating content and behavior-change content.

75. The method of claim 70, wherein the virtual content indicative of the series of the tasks to be conducted visualizes one or more virtual target objects reaching, manipulation or other addressing of which in the virtual environment or virtual part of a virtually augmented environment enables to achieve the tasks,
optionally wherein at least one virtual target object defines an optionally geometric shape, optionally a tetromino, polyomino, tetracube, polycube or alike, to be optionally manipulated by rotation, translational movement, introduction, removal, resizing or reshaping in the virtual environment or virtual part of the virtually augmented environment, optionally through conducting similar or other activity associated therewith in the physical world by the user and indicated by the measurement data.

76. The method of claim 70, comprising dynamically determining the personalized therapeutic program and/or specifically adapt the virtual content of any domain responsive to time spent by the user in using the arrangement, accessing the virtual content of the therapeutic program or selected domain(s) thereof, or generally participating in the therapeutic program.

77. The method of claim 70, wherein the user monitoring equipment comprises at least one element selected from the group consisting of: personal computer, mobile terminal, wearable electronic device, wrist device, control input sensor, accelerometer, gyroscope, inertial sensor, camera, optical sensor, location sensor, position sensor, temperature sensor, moisture sensor, pressure sensor, distance sensor, eye sensor, implantable sensor, biometric sensor, motion sensor, and a microphone.

78. The method of claim 70, comprising utilizing said user monitoring equipment, measurement data regarding the user during periods outside the consumption of the virtual content, said obtained measurement data optionally comprising at least one data element selected from the group consisting of: user activity information, call data, messaging data, communication data, physical activity or passivity data, sleep data, insomnia data, social media activity data, motion, motoric, location, position, and biometric data.

79. A computer program product optionally embodied in an optionally non-transitory computer-readable carrier medium, said program comprising instructions, which, when the program is executed by a computer, cause the computer to perform the method of claim 70.

80. A method for pain management or for treating or ameliorating kinesiophobia using the electronic arrangement according to claim 1 for the application of virtual reality (VR) or augmented reality (AR), said method comprising:
providing virtual content comprising immersive virtual environment or a virtual part of a virtually augmented environment to the user via reproduction equipment comprising a VR and/or AR projection device;
obtaining measurement data, via the user monitoring equipment of said electronic arrangement, regarding motion and/or position of the user; and
dynamically determining a personalized therapeutic program including the virtual content for representation via the reproduction equipment, based on the measurement data,
wherein the therapeutic program comprises at least two domains of different virtual content including:
(i) one or more of the domains involving behavior-change content; and
(ii) at least one other domain involving user-activating virtual content indicative of a series of tasks to be conducted by the user having regard to the virtual content through associated therapeutic behavior in the physical world outside the virtual environment or virtually augmented environment and tracked by the measurement data.

81. The method of claim 80, wherein the subject is suffering from chronic or long lasting pain.

82. A computer program product optionally embodied in an optionally non-transitory computer-readable carrier medium, said program comprising instructions, which, when the program is executed by a computer, cause the computer to carry out an embodiment of the method of claim 80.
